SEQUENCE LISTING (25 sequences)

SEQ ID NO: 1 - DNA, 13 bp, Homo sapiens
gcagagccccgcc

SEQ ID NO: 2 - DNA, 18 bp, Homo sapiens
aaacacgatggccaggac

SEQ ID NO: 3 - DNA, 24 bp, Homo sapiens
ggccttaccacctcctcaggagtc

SEQ ID NO: 4 - DNA, 38 bp, Homo sapiens
gtgcaccatggtgtctgtttgaggttgctagtgaacac

SEQ ID NO: 5 - DNA, 24 bp, Homo sapiens
ggccttaccacctcctcaggagtc

SEQ ID NO: 6 - DNA, 94 bp, Homo sapiens
aacctgccaytttcttctctttttacaatgcagtttcdacataacattggtagagtaaacaacaaaccacaagcctaaatgatgaatggtgcgc

SEQ ID NO: 7 - DNA, 17 bp, Homo sapiens
gaagaaaatggcaggtt

SEQ ID NO: 8 - DNA, 31 bp, Homo sapiens
ccaatgttagtcgaaactgcattgtaaaaag

SEQ ID NO: 9 - DNA, 43 bp, Homo sapiens
aatgttatgtcgaaactgcattgtaaaaagagaataaaatggc

SEQ ID NO: 10 - DNA, 30 bp, Homo sapiens
aatgttatgtcgaaactagaagaaaatggc

SEQ ID NO: 11 - DNA, 43 bp, Homo sapiens
gcgcaccattcatcatttaggcttgtggtttgttgtttactct

SEQ ID NO: 12 - DNA, 217 bp, Homo sapiens
gcagagccccgccgtgggtccgccygctgaggcgcccccagccagtgcgcyacctgccgagactgcgcgccatgggcaacccgggaacggcagcgccttcttgctggcacccaatdgaagccatgcgccggaccacgacgtcacgcagsaaagggacgaggtgtgggtggtgggcatgggcatcgtcatgtctctcatcgtcctggccatcgtgttt

SEQ ID NO: 13 - DNA, 26 bp, Homo sapiens
ccgcctgctgaggcgcccccagccag

SEQ ID NO: 14 - DNA, 23 bp, Homo sapiens
gcgctcacctgcaatggaagcca

SEQ ID NO: 15 - DNA, 25 bp, Homo sapiens
cccaatggaagccacgcaggaaagg

SEQ ID NO: 16 - DNA, 41 bp, Homo sapiens
acgaggtgtgggtggtgggcatgggcatcgtcatgtctctc

SEQ ID NO: 17 - DNA, 39 bp, Homo sapiens
ccgccygctgaggcgcccccagccagtgcgctyacctgc

SEQ ID NO: 18 - DNA, 14 bp, Homo sapiens
cccaatdgaagcca

SEQ ID NO: 19 - DNA, 90 bp, Homo sapiens
cgcagsaaagggacgaggtgtgggtggtgggcatgggcatcgtcatgtctctcccgcctgctgaggcgcccccagccaggcgctcacctg

SEQ ID NO: 20 - DNA, 14 bp, Homo sapiens
cccaatggaagcca

SEQ ID NO: 21 - DNA, 52 bp, Homo sapiens
cgcaggaaaggacgaggtgtgggtggtgggcatgggcatcgtcatgtctctc

SEQ ID NO: 22 - DNA, 211 bp, Homo sapiens
agtcagggcagagccatctattgcttacatttgcttctgacacaactgtgttcactagcaacctcaaacagacaccatggtgcacctgactcctgaggagcacaagtgatcgttggagtttgtctgtggtaccacgtgctgaggactcctcaagtctgccgttactgccctgtggggcaaggtgaacgtggatgaagttggtggtgaggcc

SEQ ID NO: 23 - DNA, 65 bp, Homo sapiens
caccattccggctgggcaggttggtatcaaggttacaagacaggtttaaggagaccaatagaaac

SEQ ID NO: 24 - DNA, 11 bp, Homo sapiens
ggccttaccac

SEQ ID NO: 25 - DNA, 13 bp, Homo sapiens
ctcctcaggagtc

(19) United States
(12) Patent Application Publication
(10) Pub. No.: US 2006/0183136 A1
(43) Pub. Date: Aug. 17, 2006

(54) Method for haplotyping and genotyping by melting curve analysis of hybridization probes

(76) Inventors: Genevieve Pont-Kingdon, Salt Lake City, UT (US); Elaine Lyon, Salt Lake City, UT (US); John G. Ward, Salt Lake City, UT (US)

Correspondence Address: Christopher L. Wight;Holland & Hart, P.O. Box 11583, Salt Lake City, UT, 84147-0583, US

(21) Appl. No.: 11/268,433

(22) Filed: Nov. 7, 2005

Related U.S. Application Data
(60) Provisional application No. 60/625,664, filed on Nov. 5, 2004.

(51) Int. Cl. C12Q 1/68; C07H 21/04

(52) U.S. Cl. 435/6; 536/24.3

(57) ABSTRACT

The present invention is directed to nucleic acid probes, complexes and methods of using such probes and complexes for molecular haplotyping and genotyping of mutations, using melting curve analysis of nucleic acid probes to discriminate between and determine the identity of multiple alleles at one or more loci.

### CROSS-REFERENCE TO RELATED APPLICATIONS

This application claims benefit of priority to U.S. Provisional Patent Application No. 60/625,664, filed Nov. 5, 2004, the contents of which are incorporated herein in its entirety.

### BACKGROUND OF THE INVENTION

The present invention generally relates to the field of nucleic acid chemistry. More specifically, the invention relates to hybridization probes and methods of using such probes to determine haplotypes and genotypes.

Genetic research has shown that variations or polymorphisms in a gene may cause disease, increase risk of disease or affect response to therapeutic treatment of the disease. Although polymorphisms at a single genetic locus are known to be causative, recent data show that the most common cause of disease, risk of disease or response to therapeutic treatment may actually be polymorphisms at multiple genetic loci. The genetic sequence at a particular genetic locus is referred to as the “genotype,” while the particular combination of genetic sequences or polymorphisms at multiple loci is referred to as the “haplotype.” Identification and characterization of genotypes and haplotypes has become a primary focus of genetic research.

Determining the genotype of an individual at a particular locus is generally straightforward. Among the numerous methodologies available for determining a genotype (the genetic sequence at a single location), one particular approach currently used in molecular diagnostic testing facilities utilizes a nucleic acid hybridization probe that is complementary to one of the known alleles. Hybridization probes are typically fluorescently labeled, and mutations present in PCR products are detected by analysis of the melting profile of the hybridized probe/allele complex. For example, previously small insertion/deletion mutations (1-6 nt), have been detected by derivative melting curves using FRET hybridization probes (See, e.g., Aoshima et al., Clin Chem 46:119-122 (2000); Gundry et al., Genet Test 3:365-370 (1999); Nauck et al., Clin Chem 45:1141-1147 (1999); and von Ahsen et al., Clin Chem 46:1939-1945 (2000)). In these cases, the deletion of a few nucleotides in the template, which are unavailable for binding, reduces the stability of the probe with the template, resulting in a unique melt profile and allowing discrimination and detection of a mutation based on its unique melt profile.

Determining the haplotype of an individual is more difficult. In order to map disease genes and establish founder effects attributable to haplotypes, it is necessary to determine whether or not polymorphisms at multiple genetic loci are present on the same chromosome (the linkage phase). Eukaryotes, such as human, animals and plants, contain two copies of each gene, one on each of two chromosomes inherited from a parent (with the exceptions that the male X and Y chromosomes contain only a single copy of genes, and mitochondrial DNA is present as maternally inherited copies). Frequently, these two copies contain differences in the DNA sequence, attributable to mutations or recombination events, which may increase the risk of disease, cause disease, or render an individual more or less responsive to drug treatment. If a particular mutation in a gene is present on one of the copies of the gene but not the other, the gene is said to be heterozygous for that mutation. If both copies of the gene have the same mutation, the gene is said to be homozygous for that mutation. Certain diseases are manifest only if a gene has multiple mutations at different locations on the same chromosome (the mutations are in cis phase), while the disease is not manifest if the same mutations are present but on different chromosomes (the mutations are in trans phase). Conversely, disease can be associated with the trans phase on two mutations while the non-disease status is associated with the cis phase. If an individual is heterozygous for particular variants, it is necessary to establish whether the two mutations are in cis or in trans to correctly analyze the individual's disease risk status and provide adequate genetic counseling. Merely identifying the existence of the two mutations may not therefore provide sufficient information for clinical diagnosis or prognosis of the disease or disease risk. Although genetic polymorphisms at a single genetic locus can be easily detected using basic PCR techniques, it is significantly more complex to determine the haplotype of a locus having polymorphisms at multiple genetic loci. Traditionally, haplotyping of multiple mutations has been established by analysis of the parental lineage when available or by inference from genotypes in rare cases of homozygocity or known compound heterozygotes. Such methods, however, are costly and time consuming, and are not therefore practical for use in clinical or diagnostic situations.

A number of approaches have been developed to determine the haplotype or linkage phase of a gene using molecular methods. Because diploid organisms have two sets of chromosomes containing two copies of each gene, unambiguous determination of a haplotype of one or both copies of the chromosomes previously required that the two copies be separated prior to or during analysis in order to identify which mutations are present on each of the two different chromosomes. The two diploid copies can be separated prior to analysis by transferring single chromosomes in hybridoma cell lines, gene cloning in microorganisms, dilution of DNA to single copy or analysis of single DNA molecules. These haplotyping technologies are impracticable and cost prohibitive for clinical applications, and have rarely been applied to clinical testing because the methods are complex, labor intensive, rely on extreme dilution of DNA and are often not sufficiently accurate to determine haplotypes from specific individuals.

Molecular methods have been developed for haplotyping a gene with multiple polymorphisms located short distances from each other. One particular approach has been to use allele specific amplification by PCR, a relatively easy method that has generally been usefully applied to haplotyping of individual samples. This approach has the disadvantage that it relies on very stringent reaction conditions to allow the selective amplification of only one allele.

Molecular methods have also been developed for determining the linkage phase of two distantly located alleles is disclosed by McDonald et al., Pharmacogenetics 12:93-99 (2002). First, long range PCR is used to amplify the region of the gene containing both polymorphic loci, followed by post PCR intramolecular ligation (circularization) to bring the polymorphisms into close proximity. The haplotype of the two polymorphisms, now in close proximity, is then established using allele specific PCR. This two-step approach has the disadvantage of requiring additional post-PCR steps prior to analysis, and is susceptible to intermolecular ligation between molecules, which can confound results.

Another method for molecular haplotyping, using allele-specific sequencing by a method known as pyrosequencing, has been disclosed by Odeberg et al, Biotechniques 33:1140-1108 (2002). This technique allows analysis of two consecutive polymorphisms present on a PCR product. These polymorphisms have to be in close proximity (30 nt) to allow the reaction to proceed. Additionally the size of the PCR fragment carrying the polymorphic sites is limited in order for it to be an efficient template for pyrosequencing.

Advances in the field of human genome mapping, the search for complex disease determinants, pharmacogenomics and accumulation of data from mutation screening programs emphasize the need to develop additional efficient and cost-effective methods for direct molecular haplotyping, without relying on family pedigree analysis, cloning or complex instrumentation.

### BRIEF DESCRIPTION OF THE INVENTION

The present invention is directed to probes, complexes and methods designed to facilitate haplotyping and genotyping of polynucleotide templates. In general, the present invention is based on the novel discovery that probes having multiple contiguous binding regions complementary to corresponding non-contiguous binding regions on a polynucleotide template can hybridize to the template and dissociate as a unit at a given temperature with a distinctive melting curve profile. The temperature at which the probe and template dissociates depends on the degree of complementarity between the probe binding regions and the corresponding template binding regions, and, in the case where the binding regions of the chimeric probe correspond binding regions in a template that flank insertion or deletion mutations, the dissociation temperature also depends on the presence or absence of insertion or deletion mutations between the binding regions of the template. The melting curve signature can therefore be correlated with the identity of polymorphisms within the binding regions or between the binding regions. The probes, complexes and methods of the present invention can be used to determine the identity of a plurality of mutations either on the same polynucleotide template (where the binding regions of the probe correspond to binding regions on the same template), or on separate polynucleotide templates (where the binding regions of the probe correspond to binding regions on different templates).

The present invention is thus directed to nucleic acid probes, complexes and methods of using such probes and complexes for molecular haplotyping and genotyping of mutations, using melting curve analysis of nucleic acid probes to discriminate between and determine the identity of multiple alleles at one or more loci. More particularly, the present invention is directed to nucleic acid probes, complexes and methods for using the probes and complexes, wherein the probe comprises regions that are capable of hybridizing to one of the alleles at one or more multi-allelic loci.

Generally, the present invention is directed to a chimeric nucleic acid probe for determining a haplotype or genotype of one or more polynucleotide templates having at least one genetic locus characterized by multiple alleles, wherein the probe comprises two or more contiguous binding regions, each binding region being capable of hybridizing to corresponding non-contiguous binding regions of the polynucleotide templates that encompass the genetic locus.

The present invention is also directed to a nucleic acid complex for determining a haplotype or genotype of one or more polynucleotide templates each having at least one genetic locus characterized by multiple alleles. The complex comprises a nucleic acid probe hybridized to a polynucleotide template, wherein the probe comprises two or more contiguous binding regions, each binding region encompassing a genetic locus and being capable of hybridizing to corresponding non-contiguous binding regions of the polynucleotide templates. In another embodiment, the binding regions of the polynucleotide templates include the genetic locus characterized by multiple alleles.

In another embodiment, the present invention is directed to a method for determining a haplotype or genotype of one or more polynucleotide templates each having at least one genetic locus characterized by multiple alleles, comprising:
(a) providing a nucleic acid probe comprising two or more contiguous binding regions, each binding region encompassing a genetic locus and being capable of hybridizing to corresponding non-contiguous binding regions of the polynucleotide templates;
(b) hybridizing the probe to the alleles to form a complex of the probe and the alleles;
(c) dissociating the complex and determining the melting curve profile of the complex; and
(d) correlating the melting curve profile of the complex with a melting curve profile characteristic of the alleles at each genetic loci, thereby determining the genotype of the allele at each genetic loci.

Also contemplated within the present invention is a method for determining a haplotype or genotype of a polynucleotide template having a genetic locus having multiple alleles characterized by the presence or absence of an insertion or deletion of nucleic acids in the nucleic acid template, comprising:
(a) providing a chimeric nucleic acid probe comprising two or more contiguous binding regions, each binding region being capable of hybridizing to corresponding non-contiguous binding regions of the polynucleotide templates flanking the genetic locus;
(b) hybridizing the probe to the template to form a complex of the probe and the template;
(c) dissociating the complex and determining the melting curve profile of the complex; and
(d) correlating the melting curve profile of the complex with a melting curve profile characteristic of the presence or absence of the insertion or deletion of nucleic acids, thereby detecting the presence or absence of the insertion or deletion.

In some embodiments of the invention, the genetic locus characterized by multiple alleles is located within the binding regions of the polynucleotide templates. In particular embodiments, at least a portion of the binding region of the probe is exactly complementary to one of the known alleles of the genetic locus within the binding regions of the polynucleotide template. In other embodiments, the probe need not include sequence that is exactly complementary to one of the alleles, and the probes can be slightly different from the known alleles, provided that the differences between the probe and the known alleles are sufficiently distinct that the melting curve profiles of the probe with the various alleles are distinctive and can be discriminated from each other. In other embodiments, the genetic locus characterized by multiple alleles is located outside the actual binding regions of the polynucleotide templates, but is between and flanked by the binding regions. In these embodiments, the presence or absence of insertion or deletion mutations between the binding regions has been found to have a distinctive melting curve profile that is indicative of the presence or absence of the insertion or deletion mutation.

In other embodiments, the probes include at least two binding regions that are capable of hybridizing to corresponding non-contiguous binding regions located on separate nucleic acid templates. In yet other embodiments, the binding regions of the probe are capable of hybridizing to corresponding non-contiguous binding regions located on a single nucleic acid template. The probe may comprises two, three or more contiguous binding regions.

In yet another embodiment, the nucleic acid probes may have binding regions that correspond to binding regions on the nucleic acid template that include genetic loci characterized by multiple alleles that define a haplotype.

The present invention, and other particular embodiments, are described in more detail below.

### BRIEF DESCRIPTION OF THE FIGURES

FIGS. 1-6 relate to example 1. FIG. 7 relates to Example 2. FIG. 8 relates to Example 3. FIGS. 9 relate to example 4. FIGS. 10 relate to example 5. FIGS. 11 relates to example 6

FIG. 1 shows the sequences of the WIAF 1537, WIAF 1538 region, and design and sequences of the probes. (A) shows both polymorphisms for each SNPs are separated by a diagonal and in a shaded square. (B) shows bases corresponding to WIAF 1537 and WIAF 1538 incorporated in each probe are in capital letters. The * in 1537/38 hap/lpo indicate the position of the intervening sequence of the template. Fluorophores are indicated in their respective 5′ or 3′ positions and Ph indicate phosphate. (C) shows the 3 probe sets, indicated below a representation of the WIAF 1537-1538 region. Bases incorporated in the probes are indicated under each probe. The long oligonucleotide on the right represents the anchor that is common to each set

FIG. 2 shows the design for the construction of an artificial nucleic acid template comprising two multi-allelic loci separated by a region of intervening nucleotides. (A) shows the scheme used for the construction of the CA haplotype. Arrows indicate the 3′ end of long oligonucleotides and primers both represented by horizontal lines. Numbering refers to the sequences presented in Table 1 and used to identify the different oligonucleotides in materials and methods described herein. Letters above the lines represent the base incorporated into the oligonucleotides at both SNP WIAF 1537 and WIAF 1538 positions. (B) shows the scheme for construction of the artificial template series. The random intervening sequences are indicated by the dashed line. Oligonucleotide “3” contained 20 added nucleotides compared to the wild type sequence, “4” had 20 added nucleotide compared to “3” and “5” 20 added compared to “4”.

FIG. 3 shows derivative melting curves of the 4 WIAF 1537-WIAF 1538 haplotypes using different probe sets. (A) shows 3 samples and an artificial template (C1537-A1538) homozygous at each SNP locus analyzed using the “genotyping probes” set that individually identify the SNPs. (B) shows the identical samples, analyzed with the “haplotyping probe” set. (C) shows the 4 haplotypes analyzed with the “haplotyping probe/lpo” set. (D) shows a sample heterozygous at each SNP locus (C1537-T1537-A1538-G1538) analyzed with the “genotyping probe” set and the “haplotyping probe/lpo” set showing the TA and the CG haplotypes in this heterozygous sample. Derivative melting curves for the TA and the CG haplotypes are shown as controls.

FIG. 4 shows artificial templates series hybridized to the haplotyping probe/lpo” set. (A) shows derivatives melting curves of the “haplotyping probe/lpo” set hybridized to the 16 different templates. The tables indicates the melting temperatures of the probe (in ° C.) hybridized with each template. (B) shows a model of hybridization of the haplotyping probe to the different templates. The range of melting temperature obtained from the data above are shown.

FIG. 5 shows derivative melting curve analysis of artificial template mixes mimicking heterozygous samples. Premixed haplotypes (T13G with C13A or T13A with C13G diamonds) were analyzed with the “haplotyping probe/lpo”. Single haplotypes are used as controls (plain and dotted lines).

FIG. 6 shows intermolecular versus intramolecular binding of the probe on the template. In the scheme presented on top, templates (CG and TA) are in black and grey, the probe is represented by a dashed line. Melting curve analysis of the probe on a premix containing T73A and C73G templates is shown below using a plain curve. Melting curves resulting from a intermolecular event are shown by arrows. Single haplotypes are used as controls and shown as dotted lines

FIG. 7 shows haplotypes of B2AR with haplotyping probes. (A) shows SNPs characteristic to the 3 main haplotypes are presented. Numbers in parenthesis indicate the minor haplotypes identified by the same SNPs. (B) Polymorphisms at the 4 SNPs found in the B2AR gene selected region are shaded. Primers are boxed. (C) shows bases corresponding to the SNPs polymorphisms and incorporated in the probes (in capital letters). Ph indicates a phosphate group. (D) shows the 2 probe sets, indicated below a representation of the B2AR gene selected region. Bases incorporated at the SNPs loci are indicated below the probes. The interrupted line indicates the position of the nucleotides omitted in the probe and that loop out from the template upon hybridization. (E) shows derivative melting curve analysis of the Hap −20/46 probe hybridized to sarnples homozygous (dotted lines) or heterozygous (plain lines) for each haplotypes. (F) shows derivative melting curves obtained with the Hap 46/79 probe on the same samples.

FIG. 8 shows haplotyping of 3 SNPs with a single probe. (A) Shows the design of the probe and the anchor. On the template (black) position and polymorphisms of the SNPs are indicated. The 2 DNA loop are schematized not taking into account potential secondary structures. The grey lines under the templates represent the 2 anchors. The haplotyping probe is indicated by the thick line. (B) shows partial sequences of the template and sequences of the probe in the 5′ to 3′ orientation. Fluorescent labels are indicated under the probes. (C) shows nucleotides at position −20, 46 and 79 for each haplotype tested. (D) shows derivative melting curves obtained for the 3 haplotypes analyzed in channel F2. (E) shows derivative melting curves of the same 3 samples, analyzed in channel 3.

FIG. 9 shows the multiplex genotyping of the beta globin locus. (A) shows the sequence of the amplified region and probe design. PCR primers are in bold and the probes are underlined. The lpo probe is interrupted by a dotted line that represents the sequence of the template missing in the lpo probe. (B) lists the sequences characteristic of each genotype (wild-type (WT), HbS, HbC and HbE). (C) shows the derivative melting curves of the lpo probe hybridized to homozygous samples for each genotype (plain lines) and heterozygous samples indicated by a dotted line (HbC), diamonds (HbC and HbS compound heterozygous) and triangles (HbS).

FIG. 10 shows melting curves of the unlabeled lpo beta globin probe in a HR1 instrument. Only homozygous samples are shown. Area of probe melting and amplicon melting are indicated.

FIG. 11 illustrates the capability of lpo probes to detect insertion/deletionln the drawings, templates are represented with a thick black line (with and without a loop) and probes by dotted lines (black: lpo probes and grey: anchor probes). Melting stability of the lpo probes hybridized to perfectly matched template (no loop) or template that mimic the genomic sequence and contain the sequence not present in the lpo probe (loop) are compared. (A) shows the WIAF 1537-WIAF 1538 example. The drawing indicate comparison of template with different loop length. (B) shows SNPs 46 and SNP 79 of the ADR2B receptor. Effect of the presence of the loop in two of the haplotypes (2 and 4) are shown. (C) shows the “haplotyping probe/lpo” set hybridized to 5 templates with the TG haplotype and differing by the number of nucleotides. The “TG” template is a perfect mach to the probe and its sequence is given underneath the graph.

### DETAILED DESCRIPTION OF THE INVENTION

In order that the present invention may be more readily understood, certain terms are first defined. Additional definitions are set forth throughout the detailed description.

Definitions

Units, prefixes, and symbols may be denoted in their SI accepted form. Unless otherwise indicated, nucleic acids are written left to right in 5′ to 3′ orientation. Numeric ranges recited herein are inclusive of the numbers defining the range and include, and are supportive of, each integer within the defined range. Amino acids may be referred to herein by either their commonly known three letter symbols or by the one-letter symbols recommended by the IUPAC-IUBMB Nomenclature Commission. Nucleotides, likewise, may be referred to by their commonly accepted single-letter codes. Unless otherwise noted, the terms “a” or “an” are to be construed as meaning “at least one of.” The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose. In the case of any amino acid or nucleic sequence discrepancy within the application, the figures control.

The practice of the present invention will employ, unless otherwise indicated, conventional techniques of molecular biology, microbiology, recombinant DNA techniques, and nucleic acid synthesis, which are within the skill of the art. Such techniques are explained fully in the literature. Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See, e.g., Sambrook et al. Molecular Cloning: A Laboratory Manual (2d ed., Cold Spring Harbor Laboratory Press, Cold Spring Harbor, N.Y. (1989)); Nucleic acid Synthesis (M. J. Gait, ed., 1984); Nucleic Acid Hybridization (B. D. Hames & S. J. Higgins, eds., 1984); A Practical Guide to Molecular Cloning (B. Perbal, 1984); and a series, Methods in Enzymology (Academic Press, Inc.), the contents of all of which are incorporated herein by reference.

As utilized in accordance with the present disclosure, the following terms, unless otherwise indicated, shall be understood to have the following meanings:

The term “allele” means a particular polymorphism at a designated genetic locus of a nucleotide sequence that constitutes an alternate form of a gene. A multi-allelic locus refers to a genetic locus having multiple alleles or multiple polymorphisms present in the genetic sequence among a population of different individuals. A multi-allelic locus may also refer to a genetic locus having multiple alleles or multiple polymorphisms present in the two diploid chromosomes of a single individual. The phrase “genetic locus having multiple alleles” thus means that two or more alleles have been identified or exist at that particular genetic locus among the different chromosomes within a population of individuals, or among the two or more polyploid chromosomes of a single individual. The methods and probes of the present invention may therefore be used to determine the haplotype or genotype of an individual whose two diploid chromosomes are either identical at one or more particular loci (i.e., the individual is homozygous, with the same allele at corresponding loci on the two chromosomes) or are different at one or more particular loci (i.e., the individual is heterozygous, with two different alleles at corresponding loci on the two chromosomes).

The term “binding region” means a region of a polynucleotide that is sufficiently complementary to a corresponding binding region of another polynucleotide that the two polynucleotides are capable of hybridizing to each other.

The term “chimeric,” as used in reference to the probes of the present invention, means a sequence with two or more binding regions corresponding to separate binding regions of a nucleic acid template or templates.

The terms “complementary” and “complement,” as used in reference to two nucleic acid sequences, mean that when two nucleic acid sequences are aligned in anti-parallel association (with the 5′ end of one sequence paired with the 3′ end of the other sequence), the corresponding G and C nucleotide bases of the sequences are paired, and the corresponding A and T nucleotide bases are paired. Nucleic acid analog bases not commonly found in natural nucleic acids may be included in the nucleic acids of the present invention and include, for example, inosine and 7-deazaguanine. The term “substantially complementary” means that two polynucleotide sequences are either exactly or partially complementary, but are sufficiently complementary that they are capable of detectably hybridizing to and disassociating from one another.

The term “complex” means a nucleic acid molecule that is formed by the hybridization of two single stranded nucleic acid molecules, such as a nucleic acid probe that has hybridized or bound to a DNA template.

The term “contiguous,” as used in relation to the probe binding regions, means that the binding regions of the probe directly adjoin one another. The term “non-contiguous” means that the probe binding regions are separated by intervening nucleic acids or are located on separate chromosomes.

The term “corresponding,” as used herein to describe a subject nucleotide sequence in relation to a reference nucleotide sequence, means that a subject nucleotide sequence is substantially complementary to or aligned with the reference nucleotide sequence.

The term “detectable label” means any molecule, compound, complex or combination of molecules, compounds or complexes, capable of generating a signal that can be detected upon hybridization or dissociation of the probe and template. Suitable detectable labels may include, but are not limited to, radioactive labels, fluorescent labels, or dyes.

The term “encompassing,” as used in reference to a binding region “encompassing” a genetic locus, means that the genetic locus is within the sequence of nucleotides defined by the binding region or, in the case of a genetic locus of an insertion/deletion mutation, that the genetic locus is within the intervening nucleotide sequence between non-contiguous binding regions on the same polynucleotide template.

The term “gene” means a hereditary unit consisting of a DNA sequence that occupies a specific location on a chromosome and determines a particular characteristic in an organism.

The term “genotype” means the identity of a particular allele or polymorphism at a specific genetic locus. The term genotype is used to refer to a variety of polymorphisms, including, for example, single nucleotide polymorphisms, multiple adjacent nucleotide polymorphisms, deletion mutations, insertion mutations, and other polymorphisms, as defined in the above definition of polymorphism. The term “genotyping” refers to the process of determining a genotype.

The term “haplotype” means a particular combination of two or more alleles at different genetic loci on a single chromosome that are closely linked and are inherited as a unit, and that provide a distinctive genetic pattern. Allelic variants giving rise to a haplotype may be formed by various events, including, for example, spontaneous mutations or recombination events.

The term “heterozygous” means that at a specified genetic locus there exist two or more different versions of the nucleotide sequence in the two diploid chromosomes of an individual. With reference to a sample, the term “heterozygous” means that the sample has two (diploid) copies of a chromosome or nucleotide sequence which differ at a particular locus.

The terms “hybridize,” “hybridization” and “hybridizing” mean the annealing of single-stranded nucleic acid sequences by hydrogen bonding of complementary bases to form double-stranded molecules.

The terms “locus,” and its plural form “loci,” refer to a specific position(s) or discrete region(s) on a gene, chromosome, or DNA sequence. In the context of the present invention, the term locus is used to refer to a particular position or region of polynucleotide sequences of a chromosome with which are associated multiple allelic variants, or “polymorphisms,” as defined below. The terms “first locus”, “second locus” and “third locus” refer to different positions or regions of a DNA sequence, such as a chromosome, that may be contiguous or adjacent to each other or may be separated by an intervening region of polynucleotides. At each position of the first, the second and the third loci the DNA sequence has multiple polymorphic variants (referred to herein simply as “polymorphisms”) characteristic of a particular version of the DNA sequence. A “multi-loci” probe means a probe having different regions specific or complementary to multiple loci.

The terms “nucleic acid,” “polynucleotide,” “oligonucleotide,” and “DNA,” refer to primers, probes, oligomer fragments to be detected, oligomer controls, unlabeled blocking oligomers and templates, and mean polydeoxyribonucleotides (containing 2-deoxy-D-ribose), polyribonucleotides (containing D-ribose), and any other type of polynucleotide which is an N-glycoside of a purine or pyrimidine base, or modified purine or pyrimidine bases. There is no intended distinction in length between the term “DNA,” “nucleic acid”, “polynucleotide” and “nucleic acid”, and these terms are used interchangeably herein. These terms refer only to the primary structure of the molecule. Thus, these terms include double- and single-stranded DNA, as well as double- and single-stranded RNA.

The nucleic acid sequence is not necessarily physically derived from any existing or natural sequence but may be generated in any manner, including chemical synthesis, DNA replication, reverse transcription or a combination thereof. The term “nucleic acid” may refer to a polynucleotide of genomic DNA or RNA, cDNA, semisynthetic, or synthetic origin which, by virtue of its origin or manipulation: (1) is not associated with all or a portion of the polynucleotide with which it is associated in nature; and/or (2) is linked to a polynucleotide other than that to which it is linked in nature; and (3) is not found in nature.

Because mononucleotides are reacted to make nucleic acids in a manner such that the 5′ phosphate of one mononucleotide pentose ring is attached to the 3′ oxygen of its neighbor in one direction via a phosphodiester linkage, an end of a nucleic acid is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger nucleic acid, also may be said to have 5′ and 3′ ends.

The term “polymorphism” means a variant form of the nucleotide sequence of a DNA molecule, representing an alternative form of the DNA molecule. A polymorphism may occur in the form of a substitution of one nucleotide or region of polynucleotides for another nucleotide or region of polynucleotides, resulting in no net change in the number of nucleotides. Alternatively, a polymorphism may occur in the form of a deletion or insertion of one or more nucleotides or region of polynucleotides, resulting in a change in the number of nucleotides. While the term “polymorphism” is frequently used to refer to a particular variant different from a common or wild-type form of a DNA molecule (i.e., a variant that is present at a lower frequency relative to the population of organisms to which the variant relates), the term “polymorphism” is used herein to refer to any variant, including the common or wild-type variant, as well as variants present at lower frequencies. Further, the term “polymorphism” may refer to either a particular variant of a nucleotide sequence (an “allele”), or to any one of various polymorphisms associated with a particular locus of a nucleotide sequence. Thus, reference to the polymorphisms at a particular locus means that the nucleotide sequence of one chromosome of a particular individual is different from the nucleotide sequence of the other chromosome of the same individual or is different from the nucleotide sequence of a chromosome of another individual. Polymorphisms may either be benign or causative of a particular phenotypic trait, such as a mutation that gives rise to a disease condition.

The term “probe” means a defined polynucleotide fragment that is capable of hybridizing to a complementary nucleic acid template to form a double stranded complex, due to complementarity of nucleotide sequence in the probe with a nucleotide sequence in the template. A probe typically contains a detectable radioactive or chemical label enabling detection of the probe by any of various means known to those in the art. As used herein, the term “probe” specifically refers to a polynucleotide fragment that is blocked at the 3′ end, for example, with a 2′-,3′-dideoxynucleotide, with a phosphate group, or with any other chemical moiety that blocks or removes free 3′ hydroxyl group necessary for primer extension. As used herein, the term “probe” does not therefore encompass nucleic acid primers.

The term “region” is used herein means a defined length of nucleotide sequence comprising one or more nucleotides.

The term “template” means a nucleic acid sequence to which a complementary or partially complementary nucleic acid probe hybridizes to form a double stranded nucleic acid complex.

Generally, the present invention is directed to probes, complexes and methods designed to facilitate haplotyping and genotyping of polynucleotide templates. In general, the present invention is based on the novel discovery that chimeric probes having multiple contiguous binding regions complementary to corresponding non-contiguous binding regions on a polynucleotide template can hybridize to the template and dissociate as a unit at a given temperature with a unique melting curve signature. The temperature at which the probe and template dissociates depends on the degree of complementarity between the probe binding regions and the corresponding template binding regions, and, in the case where the binding regions of the chimeric probe correspond binding regions in a template that flank insertion or deletion mutations, the dissociation temperature also depends on the presence or absence of insertion or deletion mutations between the binding regions of the template. The melting curve signature can therefore be correlated with the identity of polymorphisms within the binding regions or between the binding regions. Thus, the present invention is particularly directed to nucleic acid probes, complexes of nucleic acid probes and templates, and methods of using such probes and complexes for molecular haplotyping and genotyping, using melting curve analysis of nucleic acid probes to discriminate between and determine the identity of multiple alleles at two or more loci. In a particular embodiment, the present invention is directed to nucleic acid probes, complexes and methods for using the probes and complexes, wherein the probe comprises regions that correspond to one or more of the alleles at two or more multi-allelic loci.

The methods and materials of the present invention can be used to determine the identity of a plurality of mutations either on the same polynucleotide template, or on separate polynucleotide templates. The methods and materials of the present invention may be used, for example, to determine multiple genotypes (or haplotypes) on different polynucleotide templates using a bridging probe. The methods and materials of the invention may also be used to determine multiple genotypes on a single polynucleotide template.

The methods and materials of the invention may also be used to determine the haplotype or genotype of an allele characterized by an insertion/deletion on a single polynucleotide template, using melting curve analysis of nucleic acid probes to discriminate between and determine the identity of insertion or deletion mutations in a single polynucleotide template. In a particular embodiment, the present invention is directed to nucleic acid probes, complexes and methods for using the probes and complexes, wherein the probe comprises regions that flank a genetic locus characterized by an insertion or deletion. Where the methods and materials of the present invention are utilized to detect an insertion or deletion mutation, the binding regions of the probe may be complementary to corresponding binding regions of the polynucleotide template that do not comprise any polymorphisms. Alternatively, the binding regions of the template may comprise polymorphisms. In the event that the binding regions of the template comprise polymorphisms, the probes, complexes and methods of the invention may be utilized to simultaneously detect the presence or absence of the insertion/deletion mutation between the template binding region, but also the particular allele or alleles within the template binding region.

The present invention is particularly useful for determining the molecular haplotype (the particular combination of multiple alleles on a single gene) in a diploid DNA sample using melting curve analysis of hybridization probes that bind to the alleles defining the haplotype. In accordance with this method, a sample containing a polynucleotide template is provided in order to determine whether the DNA of the individual from whom the sample was obtained is heterozygous (i.e., has multiple alleles) or is homozygous (i.e., has only one allele) at two or more genetic loci.

The present invention is directed to a method of using a single nucleic acid hybridization probe with contiguous or adjacent first and second binding regions that are substantially complementary and hybridize to, respectively, separate or non-contiguous binding regions of a nucleic acid template. The binding regions of the nucleic acid template encompass at least one genetic locus having multiple alleles or polymorphic variants. For example, the first binding region of the probe may hybridize to a first genetic locus on the polynucleotide template having multiple alleles or polymorphic variants, and the second binding region one binding region may hybridize to a second genetic locus having multiple alleles or polymorphic variants. The first and second regions of the template are not contiguous, and are either separated by a region of polynucleotides with respect to which there are no complementary nucleotides in the nucleic acid probe, or are located on separate polynucleotide templates, for example, on separate chromosomes. The hybridization probe (with its adjacent first and second regions in close proximity) binds to the first and second regions of the template, which are brought together in close proximity to form a probe/template complex, thereby forcing the region of the template with respect to which there are no complementary nucleotides in the nucleic acid probe to “loopout”. The single hybridization probe is used to determine the identity and phase of the two alleles present on the template, using melting curve analysis of the hybridization probe. Briefly, to use melting curve analysis, a hybridization probe is labeled with a detectable label (for example, end-labeled with fluorophores that allow Fluorescence Resonance Energy Transfer when the probe hybridizes to the non-contiguous regions of the DNA template). Melting curve analysis is used to determine the melting temperature (Tm) of the probe/template complex, which will vary according to the stability of the probes with its template. Because a probe having a different degree of complementarity will have a different Tm and a different melting curve profile (for example, a probe exactly complementary to one particular polymorphic variant will have a higher Tm than a probe that differs from the same polymorphic variant by one or more nucleotides), a particular Tm will can be correlated to and be indicative of a particular polymorphic variant. Thus, the probes, complexes and methods of the present invention can be used, in conjunction with melting curve analysis, to determine haplotypes or genotypes of polymorphic variants separated by a intervening sequence

The present invention may be practiced in accordance with any one of the following various embodiments, which are provided as illustrative and not limiting examples.

In one embodiment, the present invention is directed to a chimeric nucleic acid probe for determining a haplotype or genotype of one or more polynucleotide templates each having at least one genetic locus characterized by multiple alleles, wherein the probe comprises two or more contiguous binding regions, each binding region encompassing a genetic locus and being capable of hybridizing to corresponding non-contiguous binding regions of the polynucleotide templates. In another embodiment, the binding regions of the polynucleotide templates include the genetic locus characterized by multiple alleles. Since the binding regions of the polynucleotide template correspond directly to the contiguous binding regions of the probe, and the polynucleotide template binding regions are non-contiguous, the probes of the present invention will lack nucleotide sequence complementary to regions of the polynucleotide templates that are outside of or adjacent to the polynucleotide template binding regions.

The present invention is also directed to a nucleic acid complex for determining a haplotype or genotype of one or more polynucleotide templates each having at least one genetic locus characterized by multiple alleles. The complex comprises a nucleic acid probe hybridized to a polynucleotide template, wherein the probe comprises two or more contiguous binding regions, each binding region encompassing a genetic locus and being capable of hybridizing to corresponding non-contiguous binding regions of the polynucleotide templates. In another embodiment, the binding regions of the polynucleotide templates include the genetic locus characterized by multiple alleles.

In yet another embodiment, the present invention is directed to a method for determining the haplotype or genotype of one or more polynucleotide templates each having at least one genetic locus characterized by multiple alleles, comprising:
(a) providing a nucleic acid probe comprising two or more contiguous binding regions, each binding region encompassing a genetic locus and being capable of hybridizing to corresponding non-contiguous binding regions of the polynucleotide templates;
(b) hybridizing the probe to the alleles to form a complex of the probe and the alleles;
(c) dissociating the complex and determining the melting curve profile of the complex; and
(d) correlating the melting curve profile of the complex with a melting curve profile characteristic of the alleles at each genetic loci, thereby determining the genotype of the allele at each genetic loci.

Also contemplated within the present invention is a method for determining the haplotype or genotype of a polynucleotide template having a genetic locus having multiple alleles characterized by the presence or absence of an insertion or deletion of nucleic acids in the nucleic acid template, comprising:
(a) providing a chimeric nucleic acid probe comprising two or more contiguous binding regions, each binding region being capable of hybridizing to corresponding non-contiguous binding regions of the polynucleotide templates flanking the genetic locus;
(b) hybridizing the probe to the template to form a complex of the probe and the template;
(c) dissociating the complex and determining the melting curve profile of the complex; and
(d) correlating the melting curve profile of the complex with a melting curve profile characteristic of the presence or absence of the insertion or deletion of nucleic acids, thereby detecting the presence or absence of the insertion or deletion.

As illustrated in the examples below, the methods and materials of the present invention may be used to determine the identity of a particular allele of a genetic locus. In accordance with the present invention, the probe includes a plurality of binding regions that “encompass” the genetic locus characterized by multiple alleles. In functional terms, a probe (including its binding regions) “encompasses” a genetic locus if dissociation of the probe from the template or templates to which it hybridizes is capable of providing a melting curve profile that is distinctive of the genetic locus. In structural terms, a probe (including its binding regions) “encompasses” a genetic locus if the genetic locus falls under the binding region of the probe (i.e., if the genetic locus is within the corresponding binding region of the template) or, alternatively, in the case of a genetic locus of an insertion or deletion mutation, if the genetic locus is located between and is flanked by the non-contiguous binding regions of the template to which the corresponding binding regions of the probe hybridize. Thus, in certain embodiments of the invention, the genetic locus characterized by multiple alleles is located within (i.e., among the nucleotides defining) the binding regions of the polynucleotide templates. In other embodiments, the genetic locus characterized by multiple alleles is located outside the binding regions of the polynucleotide templates and is located within the sequence between the binding regions of the template.

In some embodiments of the invention where the binding region of the probe covers the genetic locus of interest, at least a portion of the binding region of the probe is complementary to one allele of the genetic locus within the binding regions of the polynucleotide template. In such embodiments, the probe includes nucleotide sequence that is preferably exactly complementary to one of the alleles of the genetic locus. However, it is understood that the probe need not include sequence that is exactly complementary to one of the alleles. Thus, in other embodiments, the probes may be slightly different from or only substantially complementary to the known alleles, provided that the probes are capable of hybridizing to the template binding regions and the melting curve characteristics of probe and the known alleles are sufficiently distinct that the melting curve profiles enable determination of the specific allele found on the template.

In some embodiments, the present invention includes probes and methods of using probes having binding regions that are capable of hybridizing to corresponding non-contiguous binding regions located on a single nucleic acid template, such as a single chromosome. The nucleic acid probe according to the present invention may include binding regions that correspond to binding regions on the nucleic acid template that include genetic loci characterized by multiple alleles that define a haplotype. In addition, the nucleic acid probes of the invention may include binding regions that correspond to binding regions on the nucleic acid template that encompass genetic loci characterized by multiple alleles that define a haplotype, wherein one of the genetic loci is located between the template binding regions and is characterized by an allele consisting of an insertion or deletion mutation.

Determination of genotypes on a single chromosome may include determination of two genotypes are separate locations, a single genotype characterized by an insertion or deletion of nucleotide sequence, or any combination of the above. As described above, the methods and materials of the invention may be used to identify genotypes either falling within a binding region of the template or templates corresponding to one or both of the binding regions of the probe, or falling between the binding regions of a single template. The present invention contemplates combinations of the above, for example, a probe and methods of using a probe that includes two binding regions corresponding to two polymorphic sites that flank a third insertion deletion site. Also contemplated is a probe and methods of using a probe that include two binding sites, only one of which corresponds to a polymorphic site, but where the two binding sites flank a second insertion/deletion site.

Generally, the present invention is directed to a chimeric nucleic acid probe for determining the haplotype or genotype of one or more polynucleotide templates, wherein the probe comprises two or more contiguous binding regions, each binding region being capable of hybridizing to corresponding non-contiguous binding regions of the polynucleotide templates. In a particular embodiment, the probes include at least two binding regions that are capable of hybridizing to corresponding non-contiguous binding regions located on separate nucleic acid templates.

In particular embodiments, the invention is directed to a nucleic acid probe for determining the genotype of a nucleic acid template having multiple alleles at two or more loci and a region of polynucleotides between each loci, wherein the probe comprises regions of polynucleotide sequence substantially complementary to and capable of hybridizing to a corresponding region comprising one of the alleles at each loci and wherein the probe lacks nucleotide sequence corresponding to at least a portion of the region of polynucleotides between each loci.

In one aspect, the present invention is directed to a nucleic acid probe for determining the genotype or haplotype of a nucleic acid template having multiple alleles at two or more loci and a region of polynucleotides between each loci, wherein the probe comprises regions of polynucleotide sequence substantially complementary to a corresponding region of the template comprising one of the alleles at each loci and wherein the probe lacks nucleotide sequence complementary to at least a portion of the region of polynucleotides between each loci.

In another aspect, the present invention is directed to a nucleic acid probe for determining the genotype of a nucleic acid template having multiple alleles at a first locus, multiple alleles at a second locus and a region of polynucleotides between the first and second locus, wherein the probe comprises: a first polynucleotide sequence substantially complementary to a corresponding region of the template comprising one of the alleles of the first locus, a second polynucleotide sequence substantially complementary to a corresponding region of the template comprising one of the alleles of the second locus, and wherein the probe lacks nucleotide sequence corresponding to at least a portion of the region of polynucleotides between the first and second locus of the template.

In yet another aspect, the present invention is directed to a nucleic acid probe for determining the genotype of a nucleic acid template having multiple alleles at a first locus, multiple alleles at a second locus, multiple alleles at a third locus, a region of polynucleotides between the first and second locus, and a region of polynucleotides between the second and third locus, wherein the probe comprises: a first polynucleotide sequence substantially complementary to a corresponding region of the template comprising one of the polymorphisms at the first locus, a second polynucleotide sequence substantially complementary to a corresponding region of the template comprising one of the polymorphisms at the second locus, a third polynucleotide sequence substantially complementary to a corresponding region of the template comprising one of the polymorphisms at the third locus, and wherein the probe lacks nucleotide sequence complementary to at least a portion of the region of polynucleotides between the first and second locus of the template, and the probe lacks nucleotide sequence complementary to at least a portion of the region of polynucleotides between the second and third locus of the template.

In yet another aspect, the present invention is directed to a nucleic acid complex for determining the genotype of a nucleic acid template comprising (a) a nucleic acid template having multiple alleles at two or more loci and a region of polynucleotides between each loci, hybridized to (b) a nucleic acid probe comprising regions of polynucleotide sequence substantially complementary to a corresponding region of the template comprising one of the alleles at each loci and wherein the probe lacks nucleotide sequence corresponding to at least a portion of the region of polynucleotides between each loci.

In yet another aspect, the present invention is directed to a nucleic acid complex comprising (a) a nucleic acid template comprising a first locus having multiple alleles, a second locus having multiple alleles, and a region of polynucleotides between the first and second locus, hybridized to (b) a nucleic acid probe comprising a first polynucleotide sequence substantially complementary to a corresponding region of the template comprising one of the polymorphisms at the first locus, and second polynucleotide sequence corresponding to a region comprising one of the polymorphisms at the second locus, and wherein the probe lacks nucleotide sequence corresponding to a region of polynucleotides between the first and second locus of the template.

In yet another aspect, the present invention is directed to a nucleic acid complex comprising (a) a nucleic acid template comprising multiple alleles at a first locus, multiple alleles at a second locus, multiple alleles at a third locus, a region of polynucleotides between the first and second locus, and a region of polynucleotides between the second and third locus, hybridized to (b) a nucleic acid probe comprising a first polynucleotide sequence substantially complementary to a corresponding region of the template comprising one of the alleles at the first locus, a second polynucleotide sequence substantially complementary to a corresponding region of the template comprising one of the alleles at the second locus, a third polynucleotide sequence substantially complementary to a corresponding region of the template comprising one of the alleles at the third locus, and wherein the probe lacks nucleotide sequence corresponding to at least a portion of the region of polynucleotides between the first and second locus of the template, and the probe lacks nucleotide sequence corresponding to at least a portion of the region of polynucleotides between the second and third locus of the template.

In yet another aspect, the present invention is directed to a method for determining the haplotype or genotype of a nucleic acid template having multiple alleles at two or more loci and a region of polynucleotides between each loci, comprising:
(a) providing a nucleic acid probe comprising regions of polynucleotide sequence substantially complementary to a corresponding region of the template comprising one of the alleles at each loci and wherein the probe lacks nucleotide sequence corresponding to at least a portion of the region of polynucleotides between each loci;
(b) hybridizing the probe and the template to form a probe/template complex;
(c) dissociating the probe/template complex and determining the melting curve profile of the probe/template complex; and
(d) correlating the melting curve profile of the probe/template complex with a melting curve profile characteristic of the haplotype, to thereby determine the haplotype of the template.

In yet another aspect, the present invention is directed to a method for determining the haplotype or genotype of a nucleic acid template having multiple alleles at a first locus, multiple alleles at a second locus and a region of polynucleotides between the first and second locus, comprising:
(a) providing a nucleic acid probe comprising a first polynucleotide sequence substantially complementary to a corresponding region of the template comprising one of the alleles at the first locus, a second polynucleotide sequence substantially complementary to a corresponding region of the template comprising one of the alleles at the second locus, and wherein the probe lacks nucleotide sequence corresponding to the region of polynucleotides between the first and second locus;
(b) hybridizing the probe and the template to form a probe/template complex;
(c) dissociating the probe/template complex and determining the melting curve profile of the probe/template complex; and
(d) correlating the melting curve profile of the probe/template complex with a melting curve profile characteristic of the haplotype, to thereby determine the haplotype of the template.

In yet another aspect, the present invention is directed to a method for determining the haplotype or genotype of a nucleic acid template having multiple alleles at a first locus, multiple alleles at a second locus, multiple alleles at a third locus, a region of polynucleotides between the first and second locus, and a region of polynucleotides between the second and third locus, comprising:
(a) providing a nucleic acid probe comprising a first polynucleotide sequence substantially complementary to a corresponding region of the template comprising one of the alleles at the first locus, a second polynucleotide sequence substantially complementary to a corresponding region of the template comprising one of the alleles at the second locus, a third polynucleotide sequence substantially complementary to a corresponding region of the template comprising one of the alleles at the third locus, and wherein the probe lacks nucleotide sequence corresponding to at least a portion of the region of polynucleotides between the first and second locus of the template, and the probe lacks nucleotide sequence corresponding to at least a portion of the region of polynucleotides between the second and third locus of the template;
(b) hybridizing the probe and the template to form a probe/template complex;
(c) dissociating the probe/template complex and determining the melting curve profile of the probe/template complex; and
(d) correlating the melting curve profile of the probe/template complex with a melting curve profile characteristic of the haplotype, to thereby determine the haplotype of the template.

In another aspect, the nucleic acid probes used in accordance with the present invention lack nucleotide sequence corresponding to a region of polynucleotides between each loci comprising at least 5 nucleotides.

In another aspect of the invention, the allele at at least one of the loci is a single nucleotide polymorphism. In another aspect of the invention, the alleles at two or more loci are single nucleotide polymorphisms.

In another aspect of the invention, the probe further comprises a detectable label.

DNA Template

The present invention is generally directed to a method for genotyping a region of a nucleic acid template that contains multi-allelic variants at more than one loci. The nucleic acid template will typically correspond to a nucleic acid sequence found in or endogenous to any one of a variety of biological host organisms, such as bacteria, viruses, plants, animals, or humans. The nucleic acid template may be isolated or derived from any suitable source using methods well known to those in the art. For example, the nucleic acid template may be the product of amplification from genomic DNA, using polymerase chain reaction (PCR), or other means well known to those in the art.

The nucleic acid template used in the various aspects of the present invention may be any nucleic acid sequence comprising two or more loci that make up the haplotype being investigated. As described above, one aspect of the present invention provides a nucleic acid probe for determining the haplotype of a DNA region or template, where the DNA template has polymorphisms at a first locus, polymorphisms at a second locus and a region of polynucleotides between the first and second locus. The DNA template used in the various aspects of the present invention encompasses more than one multi-allelic loci, which may define a haplotype of interest. In preferred aspect of the invention, the DNA template used in the various aspects of the present invention may encompass two, three, four, five or more multi-allelic loci, which may define a haplotype of interest.

The DNA region of interest, and the DNA template corresponding to the DNA region, which encompass more than one multi-allelic loci, also includes a region of polynucleotides that separate the loci. The region of polynucleotides separating the first and second locus may be a length of nucleotide sequence comprising one or more nucleotides of any length. The number of nucleotides separating the first and second locus may be 1, 2, 3, 4, 5, 6, 7, 8, 9, 10, 11, 12, 13, 14 or greater. In particular embodiments, the number of nucleotides separating the first and second locus may be greater than 15 nucleotides, greater than 20 nucleotides, greater than 25 nucleotides, greater than 50 nucleotides, greater than 100 nucleotides, or greater than 200 nucleotides.

As described below, the nucleic acid probe of the present invention lacks nucleotide sequence corresponding to at least a portion of the region of polynucleotides between the first and second locus of the DNA region. With reference to the DNA template, this means that the DNA template includes polynucleotide sequence with respect to which there is no complementary sequence in the probe. Consequently, the absence of complementary sequence between the probe and the DNA template causes that portion of the template (the portion having polynucleotide sequence not present in the probe) to “loopout,” allowing the region comprising the allele of the first locus to come within sufficient proximity to the region comprising the allele of the second locus so that both regions are able to simultaneously hybridize to the probe, which has corresponding nucleotide sequence of one of the alleles associated with the first and second loci.

PCR Amplification

The DNA template used in connection with the methods of the present invention will generally be obtained from a region of DNA containing the loci that define the haplotype. In one particular approach, the DNA template is amplified on a single PCR product. The DNA template may be obtained, for example, by selectively amplifying particular nucleic acid sequences from among the various polymorphic variants of such sequences, using the technique of polymerase chain reaction (or PCR). Polymerase chain reaction (PCR) is widely known in the art. For example, U.S. Pat. Nos. 4,683,195, 4,683,202, and 4,800,159; K. Mullis, Cold Spring Harbor Symp. Quant. Biol., 51:263-273 (1986); and C. R. Newton & A. Graham, Introduction to Biotechniques: PCR, 2.sup.nd Ed., Springer-Verlag (New York: 1997), the disclosures of which are incorporated herein by reference, describe processes to amplify a nucleic acid sample target using PCR amplification extension primers which hybridize with the sample target. As the PCR amplification primers are extended, using a DNA polymerase (preferably thermostable), more sample target is made so that more primers can be used to repeat the process, thus amplifying the sample target sequence. Typically, the reaction conditions are cycled between those conducive to primer hybridization, nucleic acid polymerization nucleic acids denaturation.

In the first step of the reaction, the nucleic acid molecules of the sample are transiently heated, in order to denature double stranded molecules and then cooled. Forward and reverse primers are present in the amplification reaction mixture at an excess concentration relative to the sample target. When the sample is incubated under conditions conducive to hybridization and polymerization, the primers hybridize to the complementary strand of the nucleic acid molecule to the sequence of the region desired to be amplified that is the complement of the sequence whose amplification is desired. Upon hybridization, the 3′ ends of the primers are extended by the polymerase. The extension of the primer results in the synthesis of a DNA molecule having the exact sequence of the complement of the desired nucleic acid sample target. The PCR reaction is capable of exponentially amplifying the desired nucleic acid sequences, with a near doubling of the number of molecules having the desired sequence in each cycle. Thus, by permitting cycles of hybridization, polymerization, and denaturation, an exponential increase in the concentration of the desired nucleic acid molecule can be achieved.

Other DNA templates for the present invention can be the product of allele specific PCR that have already selected one polymorphism. PCR ligation products as describe in McDonald et al., Pharmacogenetics 12: 93-99 (2002) can also be used as template. In both cases, the use of the present invention to analyze these products will increase the number of loci to be phased by the experiment.

Preparation of Nucleic Acid Templates

Any specific nucleic acid sequence can be amplified by the present process. It is only necessary that a sufficient number of bases at both ends of the sequence be known so that two primers can be prepared which will hybridize to different strands of the desired sequence and at relative positions along the sequence such that an extension product synthesized from one primer, when it is separated from its template (complement), can serve as a template for extension of the other primer into a nucleic acid of defined length. The greater the knowledge of the bases at both ends of the sequence, the greater can be the specificity of the primers for the target nucleic acid sequence, and thus the greater the efficiency of the process.

The DNA region to which the nucleic acid probes of the present invention are hybridized are derived from samples obtained from any suitable biological organism containing or presumed to contain nucleic acid, such as bacteria, viruses, plants, animals and humans. Samples may be in the form of a sample of tissue or fluid isolated from an individual or individuals, including but not limited to, skin, plasma, serum, spinal fluid, lymph fluid, synovial fluid, urine, tears, blood cells, organs, tumors, and also to samples of in vitro cell culture constituents (including but not limited to conditioned medium resulting from the growth of cells in cell culture medium, recombinant cells and cell components).

Any polynucleotide molecule, in purified or non-purified form, can be utilized as the starting nucleic acid or acids, provided it contains the sequence being detected. Thus, the process may employ, for example, DNA or RNA, including messenger RNA, which DNA or RNA may be single stranded or double stranded. In addition, a DNA-RNA hybrid which contains one strand of each may be utilized. A mixture of any of these nucleic acids may also be employed, or the nucleic acids produced from a previous amplification reaction herein using the same or different primers may be so utilized. The specific nucleic acid sequence to be amplified may be only a fraction of a larger molecule or may be present initially as a discrete molecule, so that the specific sequence constitutes the entire nucleic acid.

It is not necessary that the sequence to be amplified be present initially in a pure form; it may be a minor fraction of a complex mixture, such as a portion of the beta-globin gene contained in whole human genomic DNA, or a portion of nucleic acid sequence due to a particular microorganism which organism may constitute only a very minor fraction of a particular biological sample. The starting nucleic acid may contain more than one desired specific nucleic acid sequence which may be the same or different. Therefore, the present process is useful not only for producing large amounts of one specific nucleic acid sequence, but also for amplifying simultaneously more than one different specific nucleic acid sequence located on the same or different nucleic acid molecules if more than one of the base pair variations in sequence is present.

The nucleic acid templates may be obtained from any source, for example, from plasmids such as pBR322, from cloned DNA or RNA, or from natural DNA or RNA from any source, including bacteria, yeast, viruses, organelles, and higher organisms such as plants or animals. DNA or RNA may be extracted from blood, tissue material such as chorionic villi or amniotic cells by a variety of techniques such as that described by Maniatis et al., Molecular Cloning (1982), 280-281. The method of the present invention are particularly useful in analyzing genomic DNA.

The cells may be directly used without purification of the nucleic acid if they are suspended in hypotonic buffer and heated to about 90°-100° C., until cell lysis and dispersion of intracellular components occur, generally about 1 to 15 minutes. After the heating step the amplification reagents may be added directly to the lysed cells. This direct cell detection method may be used, for example, on peripheral blood lymphocytes and amniocytes.

The target nucleic acid contained in the sample may be in the form of genomic DNA, or alternatively may be first reverse transcribed into cDNA, if necessary, and then denatured, using any suitable denaturing method, including physical, chemical, or enzymatic means, which are known to those of skill in the art. A preferred physical means for strand separation involves heating the nucleic acid until it is completely (>99%) denatured. Typical heat denaturation involves temperatures ranging from about 80° C. to about 105° C., for times ranging from a few seconds to minutes. As an alternative to denaturation, the target nucleic acid may exist in a single-stranded form in the sample, such as, for example, single-stranded RNA or DNA viruses.

The denatured nucleic acid strands are then incubated with preselected nucleic acid primers, and, optionally, a labeled nucleic acid (referred to herein as a “probe”) for purposes of detecting the amplified sequence) under conditions that facilitate the binding of the primers and probes to the single nucleic acid strands. As known in the art, the primers are selected so that their relative positions along a complex sequence are such that an extension product synthesized from one primer, when the extension product is separated from its template (complement), serves as a template for the extension of the other primer to yield a replicate chain of defined length.

PCR amplification is performed using extension primers that span the region encompassing the polymorphic loci of interest. Extension primers must be sufficiently long to prime the synthesis of extension products in the presence of the agent for polymerization. The exact length and composition of the primer will depend on many factors, including temperature of the annealing reaction, and the source and composition of the primer. For example, depending on the complexity of the target sequence, the nucleic acid primer typically contains about 15-30 nucleotides, although a primer may contain more or fewer nucleotides. Preferably, primers will contain around 20-25 nucleotides. The primers must be sufficiently complementary to anneal to their respective strands selectively and form stable complexes.

Nucleic Acid Probe

In particular embodiments, the present invention is directed to chimeric nucleic acid probes for determining the genotype of one or more polynucleotide templates each having at least one genetic locus characterized by multiple alleles. The probes of the present invention comprise two or more contiguous binding regions that are capable of hybridizing to corresponding non-contiguous binding regions of the polynucleotide templates. The binding regions of the probe are said to “encompass” a genetic locus, in the sense that the probe binding region covers or hybridizes to a corresponding binding region of the template that includes a genetic locus characterized by the allelic variation, or, alternatively, “encompasses” a genetic locus in the sense that the probe binding region corresponds to binding regions of a single template that flank a genetic locus characeterized by an insertion or deletion mutation. Thus, in this embodiment, the probes may be used either to determine the genotype of an allelic variation within the binding region of the template (directly under the probe binding region), or to determine the genotype of an insertion or deletion mutation between the two binding regions on a single template.

In an alternative embodiment, the present invention is directed to a chimeric nucleic acid probe for determining a genotype of one or more polynucleotide templates each having at least one genetic locus characterized by multiple alleles, wherein the probe comprises two or more contiguous binding regions, each binding region encompassing a genetic locus and being capable of hybridizing to corresponding non-contiguous binding regions of the polynucleotide templates comprising one allele at each genetic locus. In this embodiment, the binding regions of the probe are designed to specifically hybridize to a corresponding binding region of the template that includes the genetic locus characterized by allelic variation. It is understood that in this embodiment the binding regions of the probe may correspond to binding regions of the template that also flank a genetic locus characterized by an insertion or deletion mutation.

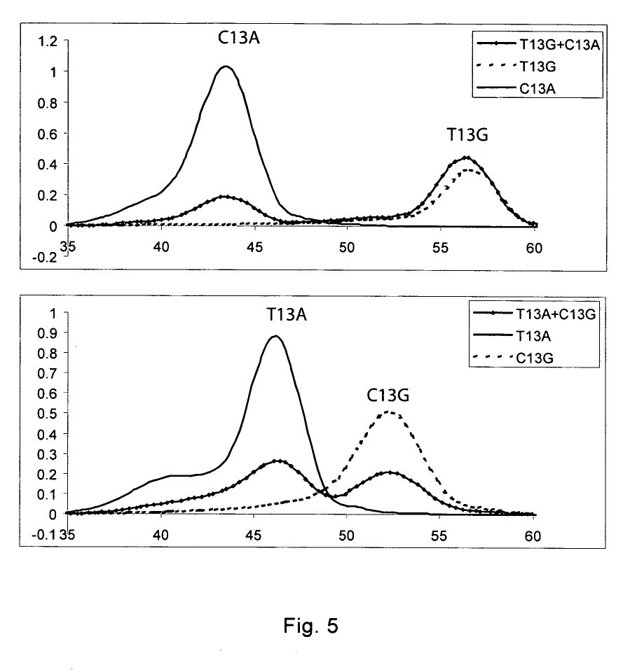

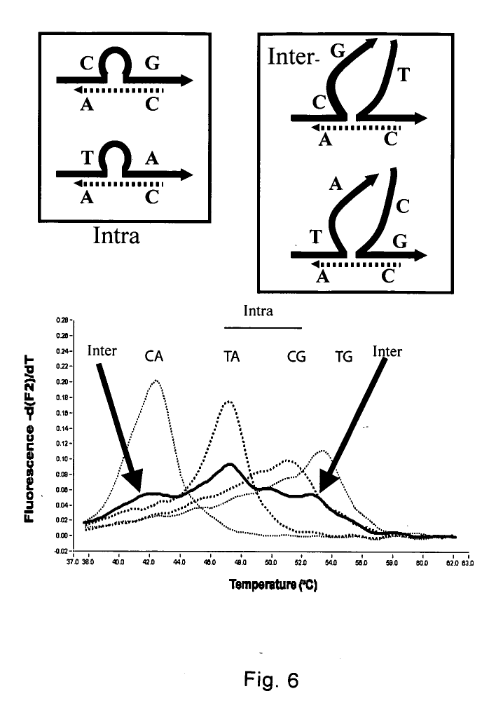

The nucleic acid probes of the present invention may also include at least two binding regions of the probe that are capable of hybridizing to corresponding non-contiguous binding regions located on separate nucleic acid templates. This embodiment is referred to herein as a “bridging” probe because it bridges two separate polynucleotide templates by simultaneously binds to the two templates, for example, two separate chromosomes. This embodiment is illustrated in the examples below, and in FIGS. 5 and 6.

The nucleic acid probe according to claim 1, wherein the binding regions of the probe are capable of hybridizing to corresponding non-contiguous binding regions located on a single nucleic acid template.

The nucleic acid probes of the present invention may include 2, 3, 4, 5 or more contiguous binding regions. In a particular embodiment, the probe comprises two contiguous binding regions. In another embodiment, the probe comprises three contiguous binding regions. In yet another embodiment, the probe comprises four contiguous binding regions. In another embodiment, the probe comprises five contiguous binding regions.

In yet another particular embodiment, the chimeric nucleic acid probes of the present invention may be defined as “consisting essentially of” two or more contiguous binding regions, the binding regions being defined as described above. A probe “consisting essentially of” two or more contiguous binding regions means that the probe includes the two binding regions, as well as any other elements or components that do not materially affect the basic and novel characteristics of the probe. The basic and novel characteristics of the probe are that it is capable of hybridizing to two non-contiguous regions of one or more polynucleotide template and dissociate as a unit, so as to yield a distinctive melting point curve signature that enables determination of a genotype encompassed by the probe. In the context of the this embodiment, it is understood that a detectable label, for example, would not be considered to materially affect the basic and novel characteristics of the probe. Similarly, it would be expected that additional nucleotide sequence or other chemical entities on the 5′ or 3′ end of the probe would not materially affect the basic and novel characteristics of the probe.

In another particular embodiment of the invention, the chimeric nucleic acid probes may be defined as “consisting of” two or more contiguous binding regions and a detectable label, the binding regions being defined as described above. A probe “consisting of” two or more contiguous binding regions and a detectable label means that the probe includes only the two binding regions and the detectable label, and no other elements or components.

In one aspect, the present invention is directed to a nucleic acid probe for determining the haplotype of a DNA region or template having multiple alleles at a first locus, multiple alleles at a second locus and a region of polynucleotides between the first and second locus. The nucleic acid probe comprises a first polynucleotide sequence substantially complementary to a corresponding region of the template comprising one of the alleles at the first locus, a second polynucleotide sequence substantially complementary to a corresponding region of the template comprising one of the alleles at the second locus, and the probe also lacks nucleotide sequence corresponding to a region of polynucleotides between the first and second locus.

In another aspect of the invention, the present invention is directed to a nucleic acid probe for determining the haplotype of a nucleic acid template having multiple alleles at three or more loci. For example, the nucleic acid template may have multiple alleles at a first locus, multiple alleles at a second locus, multiple alleles at a third locus, a region of polynucleotides between the first and second locus, and a region of polynucleotides between the second and third locus. In this case, the probe will comprise a first polynucleotide sequence substantially complementary to a region comprising one of the alleles at the first locus, a second polynucleotide sequence substantially complementary to a region comprising one of the alleles at the second locus, a third polynucleotide sequence substantially complementary to a region comprising one of the alleles at the third locus, and the probe will lack nucleotide sequence complementary to at least a portion of the region of polynucleotides between the first and second locus of the template, and the probe lacks nucleotide sequence complementary to at least a portion of the region of polynucleotides between the second and third locus of the template.

A probe that “lacks nucleotide sequence complementary to a region of polynucleotidesbetween the first and second locus” means that the nucleotide sequence of the probe does not include nucleotide sequence that is complementary to the region of polynucleotides between the first and second locus. Conversely, with reference to the template region of polynucleotides between the first and second locus, a probe that “lacks nucleotide sequence complementary to a region of polynucleotides between the first and second locus” means that the region of polynucleotides between the first and second locus of the template includes nucleotide sequence with respect to which there is no complementary nucleotide sequence in the probe. A probe may lack nucleotide sequence corresponding to a region of polynucleotides between the first and second locus because the corresponding region of polynucleotides between the first and second locus has been deleted from the probe, resulting in a probe having fewer nucleotides between the alleles compared to the template region, or alternatively because nucleotides of the probe that are aligned with nucleotides in the template are not complementary.

The nucleic acid probe comprises a first polynucleotide sequence substantially complementary to a corresponding region of the template comprising one of the alleles at the first locus and a second polynucleotide sequence substantially complementary to a corresponding region of the template comprising one of the alleles at the second locus. The polynucleotide sequence of the probe thus includes regions of nucleotides that are specific or exactly complementary to a region including at least one of the alleles of the first locus and a region including one of the alleles of the second locus. Alternatively, the polynucleotide sequence of the probe may also includes regions of nucleotides that are substantially complementary to the corresponding region of the allele, provided that the Tm of the substantially complementary probe and the different alleles is sufficiently different that they can be discriminated.

Although the size (i.e., the number of nucleotides) of the region comprising one of the polymorphisms at the first locus and the second locus may vary, it will be appreciated that a region of sufficient size is selected to enable the two polymorphic regions of the probe to hybridize to the DNA template. It will further be appreciated that the region should be of a size sufficient to enable discrimination between the melting temperature (Tm) of the hybridized complex formed by the probe to the DNA template having the polymorphism complementary to the probe polymorphism and the hybridized complex formed by the probe to the DNA template having the polymorphism not complementary to the probe polymorphism. The regions of the probe that comprise one of the polymorphisms of either the first or second loci and are complementary or substantially complementary to the corresponding region of the nucleic acid template consist of a length of nucleotide sequence comprising one or more nucleotides. The number of nucleotides in this particular region is preferably at least 5, 6, 7, 8, 9, 10, 11, 12, 13, 14 or greater. In more preferred embodiments, the region hybridizing on a give SNP will be at least 5 nucleotides, with 2 nucleotides on each side of the SNP. In particular embodiments, the number of nucleotides of the region may be greater than 15 nucleotides, greater than 20 nucleotides, greater than 25 nucleotides, greater than 30 nucleotides, or greater than 35 nucleotides.

One skilled in the art will appreciate that the size of the nucleic acid will affect the ability to discriminate between the Tm of different polymorphic variants. A region of complementarity that is too large will hybridize under very similar conditions to a region of complementarity of the same size but which differs by only a single nucleotide, since the region of non-complementarity of only a single nucleotide constitutes a relatively smaller percentage of a large region compared to a small region, and will therefore have less impact on the ability of the probe to hybridize to the DNA template. Because the method of the present invention requires the ability to discriminate between the Tm of a probe/template complex with one degree of complementarity versus another probe/template complex with a different degree of complementarity, discrimination is enhanced with use of a probe that contains nucleotides that are complementary to fewer nucleotides, such as 2, 3, 4, 5, 6, or 7 nucleotides adjacent to either the 5′ or 3′ side polymorphism.

In other preferred aspects, the probes are end-labeled with fluorophores that allow Fluorescence Resonance Energy Transfer (FRET, review in Didenko, Biotechniques 31:1106-1116, 1118, 1120-1201 (2001)), between a reference probe and an anchor probe when both probes hybridize adjacently on a DNA template.

In accordance with the above method, a hybridization probe with adjacent regions complementary to separate regions (a first locus, and a second locus) comprising two or more alleles are used to identify or determine the phase of the two alleles actually present on a DNA template.

In preferred aspects of the present invention, it is contemplated that the polynucleotide regions of the probe are exactly complementary with respect to all nucleotides of one polymorphic locus of the corresponding DNA template region. In other aspects of the invention, however, it is contemplated that the probes, nucleic acid complexes and methods of the present invention also use polynucleotide regions that are only substantially complementary to the corresponding region of the template comprising one of the alleles, such that the probe preferentially hybridizes to the template at that location.

The position of the nucleic acid probe in relation to the locus associated with an allele may vary. The polymorphic loci of the template may be located at or near either the 5′ or 3′ end, or at an internal position, of the corresponding region of the probe. In a preferred aspect of the invention, the polymorphic loci of the template may be located at or near the middle of the region of the probe, with generally an equal number of nucleotides to the 3′ and 5′ side of the polymorphism that are complementary to the corresponding region of the DNA template.

In one aspect of the invention, the nucleic acid templates differ by one or two SNPs and the presence of intervening sequences. In another aspect, the nucleic acid haplotyping probes can also discriminate and haplotype templates with variable number of two adjacent nucleotide repeats. Various combinations of polymorphisms could be distinguished using haplotyping probes.

The length separating the two SNPs was limited in the above experiments by the ability to synthesize long nucleic acids used to build templates; however, a similar approach may be used for natural polymorphisms separated by greater distances. In these cases, as described in premixed experiments, several PCR products may be bridged with one probe. Conditions for PCR should avoid PCR mediated recombination.

Template/Probe Complex

In another aspect, the present invention is directed to a nucleic acid complex comprising a DNA template and a nucleic acid probe. In a particular aspect, the invention is directed to a nucleic acid complex comprising (a) a nucleic acid template comprising a first locus having multiple alleles, a second locus having multiple alleles, and a region of polynucleotides between the first and second locus, and (b) a nucleic acid probe comprising a first polynucleotide sequence substantially complementary to a corresponding region of the template comprising one of the alleles at the first locus, and second polynucleotide sequence substantially complementary to a corresponding region of the template comprising one of the polymorphisms at the second locus, and wherein the probe lacks nucleotide sequence complementary to a region of polynucleotides between the first and second locus of the template.

In another aspect, the present invention is directed to a nucleic acid complex comprising (a) a nucleic acid template comprising a first locus having multiple alleles, a second locus having multiple alleles, and a region of polynucleotides between the first and second locus, and (b) a nucleic acid probe comprising a first polynucleotide sequence substantially complementary to a corresponding region of the template comprising one of the polymorphisms at the first locus, and a second polynucleotide sequence substantially complementary to a corresponding region of the template comprising one of the polymorphisms at the second locus, a detectable label activated by hybridization or dissociation of the probe and the template, and wherein the probe lacks nucleotide sequence complementary to a region of polynucleotides between the first and second locus of the template.

A nucleic acid complex comprising (a) a nucleic acid template comprising multiple alleles at a first locus, multiple alleles at a second locus, multiple alleles at a third locus, a region of polynucleotides between the first and second locus, and a region of polynucleotides between the second and third locus, and (b) a nucleic acid probe comprising a first polynucleotide sequence substantially complementary to a corresponding region of the template comprising one of the alleles at the first locus, a second polynucleotide sequence substantially complementary to a corresponding region of the template comprising one of the alleles at the second locus, a third polynucleotide sequence substantially complementary to a corresponding region of the template comprising one of the alleles at the third locus, and wherein the probe lacks nucleotide sequence complementary to at least a portion of the region of polynucleotides between the first and second locus of the template, and the probe lacks nucleotide sequence complementary to at least a portion of the region of polynucleotides between the second and third locus of the template.

Detection of Probes

In accordance with the present invention, the identification of the different haplotypes relies on the ability of the nucleic acid probe hybridized to a template to dissociate as a unit. Accordingly, haplotyping may be performed in accordance with the invention using other probe designs that allow genotyping by determining the Tm of a probe and template. These systems include fluorescein-labeled probes (See, e.g., Crockett et al., Anal Biochem 290:89-97 (2001), and Vaughn et al., Lab Invest 81:1575-1577 (2001)), MGB-Eclipse-probes (See, e.g., Afonina et al., Biotechniques 32:940-944, 946-949 (2002), and Kutyavin et al., Nucleic Acids Res 28:655-661 (2000)) and molecular beacon (See, e.g., Bonnet et al., Proc Natl Acad Sci USA 96:6171-6176 (1999), and Tyagi et al., Nat Biotechnol 14:303-308 (1996)). High throughput analysis could be performed using instrument such as the LightTyper (Roche Applied Science), a ABI 7900 or a Rotor-Gene (Corbett Research, Biotage). Detection of hybridization or dissociation may be performed using any available method capable of determining the Tm of the probe/template complex, including method that detect either loss or gain of signal upon either hybridization or dissociation. Detection methods may utilize methods that use radioactive labels, fluorescent labels, or dyes

Melting Curve Analysis

The haplotyping assay of the present invention is based on melting curve analysis of a probe that binds to the target sequence of a nucleic acid template amplicon. The assay exploits the thermal properties of DNA, namely melting temperature (Tm) or annealing temperature. The Tm is the temperature at which, under specified conditions, 50% of the base pairs of a nucleic acid complex have dissociated. When a fluorescently labeled sequence-specific oligonucleotide probe (typically about 20 base length) hybridizes with a target DNA sequence to form a complex, it can generate fluorescent signal. Upon heating, the probe will melt off/separate from the target sequence of the complex at its Tm, resulting in the loss of fluorescent signal. Alternatively, methods may be used that rely on a loss of signal with hybridization (i.e., gain of signal with dissociation). This change in signal as a result of dissociation (or hybridization) can be captured as a melt curve and can be converted into the derivative melt peak, from which the genotype can be derived. If the probe sequence, designed to match the wild type DNA, and the target DNA sequence are perfectly complementary to each other, the probe Tm will be high. For a mismatched (variant/mutant) complex, probe Tm will be low. This discrimination in Tm allows for discrimination between and assignment of genotypes.

The method of the present invention may be implemented using a hybridization probe set (comprising two probes: an anchor probe and a sensor probe). Hybridization probes work based on the fluorescence resonance energy transfer (FRET) principle. In this method, an fluorescein-labeled probe (donor) and a LCR-640-labeled probe (acceptor) are designed to anneal to the complementary target on the target amplicon to generate fluorescent signal. When heated, one of the annealed probes will melt off from the template, resulting in loss of fluorescent signal. The Tm of each sample is an indicator of its classification as wild type, homozygote mutant, or heterozygote. The method of the present invention may also be implemented using a simple probe, which utilizes a sequence specific single probe to generate fluorescent signal upon annealing to the target sequence. The simple probe also gives information similar to that obtained with the hybridization probe. The methods of the present invention may also be implemented using unlabeled probes in combination with a non-specific intercalating dye.

Melting curve analysis can be performed using commercially available reagents and instrumentation. For example, the LightCycler® instrument (Roche Molecular Biochemicals) enables both the amplification and the real-time, on-line detection of a PCR product, thus allowing accurate quantification to permit detection and genotyping of single nucleotide polymorphisms using melting curve analysis. During the melting curve analysis, the LightCycler instrument monitors the temperature-dependent hybridization of sequence specific hybridization probes to single stranded DNA. No post-PCR processing is needed and the risk of contamination is minimized, as amplification and genotyping are performed in the same sealed capillary without any further handling steps.

In brief, during the PCR, a DNA fragment of the respective gene is amplified with specific primers from human genomic DNA. The amplicon is detected by fluorescence using specific pairs of nucleic acid hybridization probes. Hybridization probes consist of two different oligonucleotides that hybridize to an internal sequence of the amplified fragment during the annealing phase of PCR cycles. One probe is labeled at the 5′-end with a LightCycler-Red fluorophore (for example, LightCycler-Red 640 or LightCycler-Red 705) and, to avoid extension, is modified at the 3′-end by phosphorylation. The other probe is labeled at the 3′-end with fluorescein. Only after hybridization to the template DNA do the two probes come in close proximity, resulting in fluorescence resonance energy transfer (FRET) between the two fluorophores.

During FRET, fluorescein, the donor fluorophore, is excited by the light source of the LightCycler instrument, and part of the excitation energy is transferred to LightCycler-Red, the acceptor fluorophore. The emitted fluorescence of the LightCycler-Red fluorophore is measured. These Hybridization Probes are also used to determine the genotype by means of a melting curve analysis implemented after the amplification cycles are completed and the amplicon is formed. The melting temperature (Tm) of the complex consisting of hybridization probe and single-stranded target DNA sequence is dependent on GC content, length, degree of homology and sequence order. One of the two hybridization probes covers the region of the potential mutation (mutation probe) and has a lower Tm than the adjacent probe (anchor probe). Hybridization Probe/DNA hybrids containing a mismatch melt at a lower Tm than perfectly matched probes. Hence, wildtype, mutant and heterozygous genotypes can be distinguished by different melting temperatures, displayed in the LightCycler software as melting peaks. The time required to genotype SNPs is reduced to less than 40 min for 32 research samples, while eliminating post PCR-processing and minimizing the risk of contamination. The principle of using hybridization probes and melting curve analysis to detect specific mutations and SNPs has been described in detail elsewhere by Reiser et al., Biochemica 2:12-15 (1999), and Bernard et al., Clin. Chemistry 46:147-148 (2000), the contents of which are hereby incorporated herein by reference in their entirety. Further details concerning the melting curve analysis is also available at http://www.biochem.roche.com/lightcycler.

Haplotying Using Hybridization Probes

The methods of the present invention may be used to determine haplotypes or genotypes of any DNA sequence derived from any prokaryotic or eukaryotic organism, including, but not limited to, plants or animals, in particular humans.

The method of the present invention is particularly useful for haplotyping DNA sequences derived from a diploid organisms (which have two copies of each gene, one copy inherited from each parent). The method of the present invention may also be used for haplotyping DNA sequences present in a haploid (i.e., single chromosome) organism, for example, to determine the presence, identity or haplotype of an infectious pathogen in an individual with a mixed infection of variant forms of the pathogen. Haplotyping by hybridization is particularly useful in the field of human genetics (to identify the genetic determinants of complex diseases), anthropology (to identify and test haplotypes associated with particular populations and thereby determination the origin and migration patters of human populations) and pharmacogenetics (to identify and test different haplotypes associated with different drug response).

The present invention may be used, for example, to haplotype the estrogen receptor gene ESR1. Haplotypes created by different association of three polymorphisms found in the ESR1 gene are being studied in relation to osteoporosis, cancer risk and cardiovascular diseaseThese 3 polymorphisms are markers and not causative. The three polymorphisms are a (TA)n repeat in the promoter of the gene, and two single nucleotide polymorphisms in intron 1: a T to C (Pvu II polymorphism) and an A to G (Xba I polymorphism). The promoter polymorphism is separated by 35 kb from the 2 SNPs of intron 1. Both SNP are separated by 50 nucleotides. Studies have shown that three of the four possible haplotypes using the Pvu II and the Xba I polymorphisms are found in the overall population and each of these haplotypes is associated with variable length of the TA repeat An haplotyping probe as describe in the present invention would distinguish the PvuII and Xba I haplotypes. Additionally if the template used is the result of an allele specific reaction of the TA locus, the 3 SNP would be analyzed directly and haplotype defined. Another example that can use haplotyping probes is the haplotyping of the mannose-binding lectin 2 (MBL2). This gene is involved in the response of individuals to infections. Six SNPs and the haplotypes they define generate the different structures observed in the polypeptide. A combination of allele specific PCR (or PCR followed by intramolecular ligation) and analysis of SNP in close proximity by haplotyping probes could provide a clinical molecular haplotyping assay.

Other examples include genes where the existence of simple (1 mutation), compound (2 mutations on separate chromosomes) and complex (with two or more mutations on one chromosome and a mutation in the other) genotypes have been reported, for example, MCAD, MTHFR (Tonetti et al., J Inherit Metab Dis. 24:833-42 (2001), HFE (Mullighan et al., Gut 42:566-9 (1998)).

The present invention is also useful in the field of pharmacogenetics, for use in correlating specific individual genetic polymorphisms and individual responses to specific pharmaceutical compound. Examples of haplotypes that are relevant to the field of pharmacogenetics are (but are not limited to) CYP3A4, TPMT, IL4RA (McDonald et al., Pharmacogenetics 12:93-9 (2002) and B2AR (see Example 2, below).

The present invention is also useful for haplotyping of oncogenes or tumor suppressor genes, which may be associated with cancer susceptibility. Currently, haplotypes in populations are determined using statistical methods. Molecular methods using hybridization probes could be developed to directly access some of the haplotypes from genes such as BRCA1 and BRCA2, RAD51, TP53 (Bonnen et al., Genome Res 12:1846-53 (2002)), and ESR1 (Weiderpass et al., Carcinogenesis 21:623-7 (2000)).

Molecular haplotyping assays often use allele specific PCR approaches. In certain cases where polymorphisms are located in relatively close proximity, melting curve analysis of haplotyping probes as described herein would be a useful alternative to other methods in current use. When multiple polymorphism are implicated haplotyping probes could be used as a complement to allele specific assays, reducing the number of allele specific reactions needed to determine haplotypes. Use of haplotyping probes to analyze product of ligation from long PCR would also reduce the number of steps involve in determining haplotypes of polymorphisms separated by large distances (McDonald et al., Pharmacogenetics 12:93-9 (2002). Haplotyping probes can also be designed to distinguish very closely related genes (or organisms) that differs by at least 2 polymorphisms in a 100, 200, 300, 400, 500 or more nucleotide range.

The following Examples illustrate particular embodiments and aspects of the present invention, and are not to be construed as limiting the scope of the claimed invention.

### EXAMPLE 1

### Haplotyping SNPs

The utility of the method, nucleic acid probe and probe/template complex of the present invention is illustrated in the following example, which shows that two SNPs from chromosome 21 can be haplotyped using melting curve analysis of nucleic acid hybridization probes. The first probe covers both SNPs of interest and the second one has a sequence deleted between the 2 SNPs compared to the template allowing haplotyping of SNPs further apart. Using series of “artificial” templates with increasing distance between 2 SNPs it is demonstrated that a hybridization probe will still melt as a unit and discriminates the 4 haplotypes even when the distance between SNPs is 87 nucleotides. The additional sequences (13 nucleotides to 72 nucleotides, depending on the template) must loop out or bulge to allow probe binding to the template.

SNPs Selection, Primers and FRET Probes

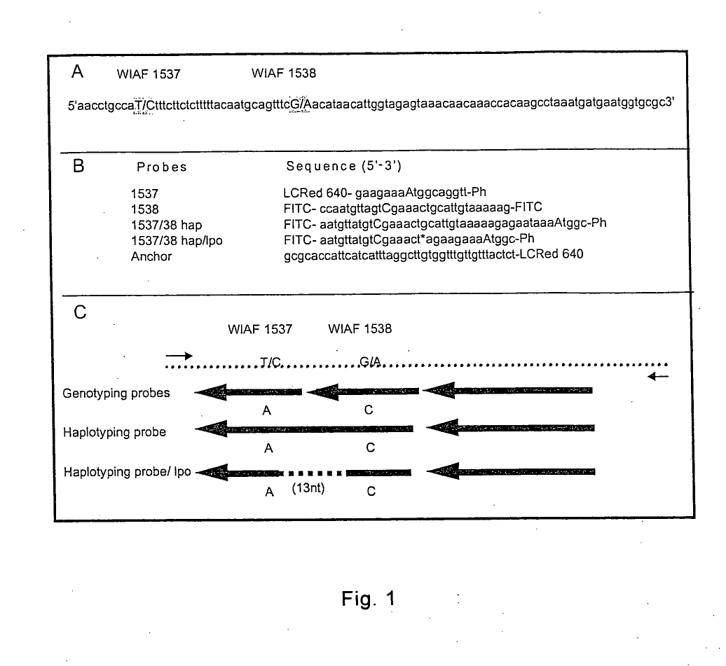

The two SNPs WIAF-1537 and WIAF-1538 on chromosome 21 were selected from the Whitehead Institute data base (http://www.genome.wi.mit.edu/SNP/human/maps/Chr 21.All.html) (Pont-Kingdon and Lyon, Clinical Chemistry 49:1087-1094 (2003)). Table 1 provides the name and sequences of all primers used in this study. Primers shorter than 30 nt were provided as desalted by the DNA-peptide core facility at the University of Utah (Salt Lake City, Utah). Overlapping long nucleic acids for the “artificial templates” with lengths greater than 50 nt and were synthesized and dHPLC purified by the same facility. Hybridization probes were designed to genotype SNPs following guidelines described previously (Lyon, Expert Rev. Mol. Diagn. 1:92-101 (2001)). Both genotyping and haplotyping probe sequences are presented in FIGS. 1. Probes labeled with fluorescein (Biogenix), LCRed640 and LCRed705 (Roche Applied Science) were synthesized by Idaho Technology (Salt Lake City, Utah).

Construction of Artificial Templates Containing WIAF 1537 and WIAF 1538 SNPs

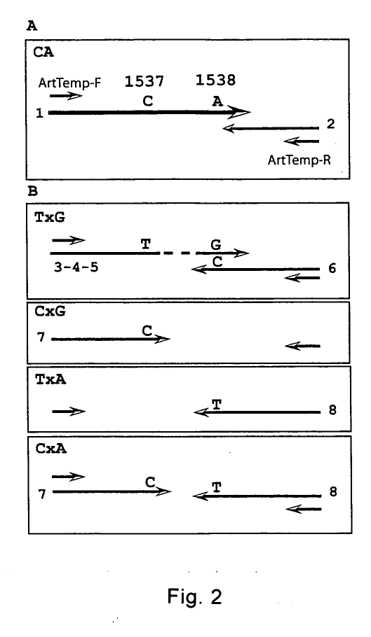

The designs used to construct all artificial templates are presented in FIG. 2. The WIAF-1537C, WIAF 1538A haplotype (CA haplotype) was created using the forward primers “ArtTemp-F” (0.5 μM) and the long primer #1 (1537C/38A-F) (0.1 μM) and the reverse primers “ArtTemp-R” (0.5 μM) and long primer #2 (1537/38-R) (0.1 μM) (FIG. 2A and Table 1). The two long primers overlap by 20 nt. PCR was performed using 1×PCR buffer (Roche Applied Science, Indianapolis, Ind.), 50 nM each dNTP, the 4 primers at concentrations indicated above and AmpliTaq DNA polymerase (Roche Applied Science). PCR was run in Perkin Elmer 2700 with the following conditions: 10′ at 94° C. followed by 5 cycles with an annealing temperature of 52° C. (96° C., 20″; 52° C., 30″; 72° C., 30″) and 35 cycles with an annealing temperature of 58° C. (96° C., 20″; 52° C., 30″; 72° C., 30″). The band sized at 153 nucleotides (nt) was cut out from agarose gel and sequenced by dye terminator.

Series of artificial templates with increased distance between the SNPs were constructed following the schema of FIG. 2B with the primers given in Table 1. The T33G, T53G and T73G templates were prepared first using primers ArtTemp-F, ArtTemp-R, the appropriate forward primers #3, #4 or #5 that contain insertions of 20, 40 and 60 random nt and the reverse primer #6 (1538G-R). Long primers and short primers were used at concentration described above. PCR was performed using PuReTaq Ready-To-Go™ PCR beads (Amersham, Piscataway, N.J.) and conditions were as follows: 1040 at 94° C. followed by 5 cycles with an annealing temperature of 62° C. (96° C., 20″; 62° C., 30″; 72° C., 30″) and 40 cycles with an annealing temperature of 60° C. (96° C., 20″; 60° C., 30″; 72° C., 30″). The “TxG” products were purified from agarose gel and 1 μl of purified products used as templates for the construction of the CxG, the TxA and the CxA haplotypes. Haplotypes CxG were constructed using Art-temp-R and the long primer #7 each at a concentration of 0.5 μM. ArtTemp-F and long primer #8 were used for the construction of the TxA haplotypes. CxA haplotypes was constructed using both ArtTemp primers and both long primers #7 and 8. All 16 artificial templates were purified using Qiagen purification kit eluted in 50 μl of water. All products were analyzed on a 2% agarose gel and sequenced to confirm the incorporation of the SNPs.

PCR, Melting Conditions and Analysis for Genotyping and Haplotyping Assays

All PCR was performed in capillary tubes with 10 μl reactions on a LightCycler Instrument (Roche Applied Science) using the 5.32 run version with automated gain adjustment.

The conditions for WIAF 1537 and WIAF 1538 amplification from genomic DNA and artificial templates were as follow: approximately 50 ng of templates were amplified in presence of 0.5 μM of each primers ArtTemp-F and ArtTemp-R, 200 μM each dNTPs, 1× “Clear Buffer 20 mM” (20 mM MgCl2, 50 mM Tris, pH 8.3, 500 mg/L bovine serum albumin, Idaho Technology), 1 μl of AmpliTaq DNA Polymerase (Roche Applied Science) premixed with TaqStart Antibody (Clontech, Palo Alto Calif.) and 0.2 μM of each appropriate hybridization probe. The following conditions were used for the reactions: denaturation at 94° C. for 2 seconds, annealing at 60° C. for 10 seconds and extension at 72° C. for 15 seconds for 45 cycles. Programmed transition rates were 20° C./second from denaturation to annealing and from extension to denaturation and 2° C./seconds from annealing to extension. The amplification cycles were followed by a melting cycle in which DNA was denatured at 95° C. with no holding time, cooled to 35° C. using a rate of 20° C./sec and held for 120 seconds. Temperature was then raised to 85° C. with a transition rate of 0.1° C./sec. Fluorescence was continuously monitored during the melt.

Melting curves were converted into negative derivative curves of fluorescence with respect to temperature (−dF/dT) by the LightCycler Data Analysis software. All analyses were performed with background correction and color compensation. Genotyping and haplotyping of WIAF 1537 and WIAF 1538 were analyzed using the F2 channel.

Samples

Samples used were from DNA de-identified following Institutional Review Board protocol. They were extracted from whole blood with the MagNa Pure LC DNA Isolation Kit I (Roche Applied Science)

Genotyping and Haplotyping of 2 SNPs in Close Proximity on Chromosome 21

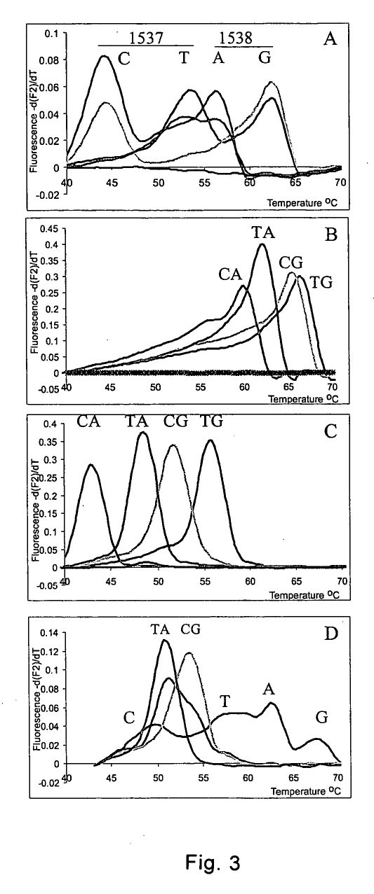

Chromosome 21's SNPs WIAF 1537 and WIAF 1538 were selected while searching for markers with high heterozygosity index on this chromosome. These two polymorphic sites are separated by 27 nt (FIG. 1A) and are genotyped independently in a multiplex reaction containing three nucleic acids labeled with FITC and LCRed 640 (FIG. 2B&C-genotyping probes: “1537”, “1538” and “Anchor”). The SNPs identity are determined by the different melting temperatures of the specific probes: FIG. 3A shows 4 samples homozygous for each SNP. Two melting curves are present per sample; one indicates the genotype of WIAF 1537 and the other indicates the genotype of WIAF 1538 independently. In each case, the higher stability is observed for the allele with a perfect match with the probes (WIAF 1537T and WIAF 1538G). The TG, CG, and TA samples were from a random DNA collection while the CA sample, containing the less common alleles (WIAF 1537C and WIAF 1538A, data not shown) was artificially constructed. Obviously since these samples are homozygous for both these samples contain single haplotypes: For example, on both chromosomes of the TG sample the WIAF 1537T allele is associated with the WIAF 1538G allele. This haplotype will be refer as “T-G” in the rest of the paper. To determine directly the haplotypes we designed a single probe that overlaps both SNP positions (FIGS. 2B and 2C, haplotyping probe: “1537/38 hap”) with each SNP a perfect match to the T-G haplotype. The use of this probe reduces the 2 derivative melting curves to a single curve per sample corresponding to definite haplotypes (FIG. 3B). If an internal deletion (13 nucleotides) between both SNP is created in the probe (FIGS. 1B and 1C, haplotyping/loop out probe: “1537/38 hap/lpo”) compared to the template, the resolution of the 4 haplotypes is enhanced (FIG. 3C). The observation of only one derivative melting curve per haplotype and the clear distinction between them indicates that the probe acts as a unit and does not melt from the template in two different domains. The 13 extra nucleotides present in the template must bulge or loop out (lpo) to allow continuous binding of the probe. In FIG. 3D, a sample heterozygous for both WIAF 1537 and WIAF 1538, as shown by the hybridization profile with the SNP specific independent probes (4 melting curves, C, T, A, G), was haplotyped with the haplotyping probe (hap/lpo). Comparison of the melting profile (black line, not marked) with the single haplotypes controls (TA and CG) indicates that both the TA and the CG haplotypes are present in this sample.

Various Lengths between SNPs

It was presumed that the length between the SNPs could affect the stability of the haplotyping probe and therefore the discrimination of haplotypes in samples heterozygous at both loci.

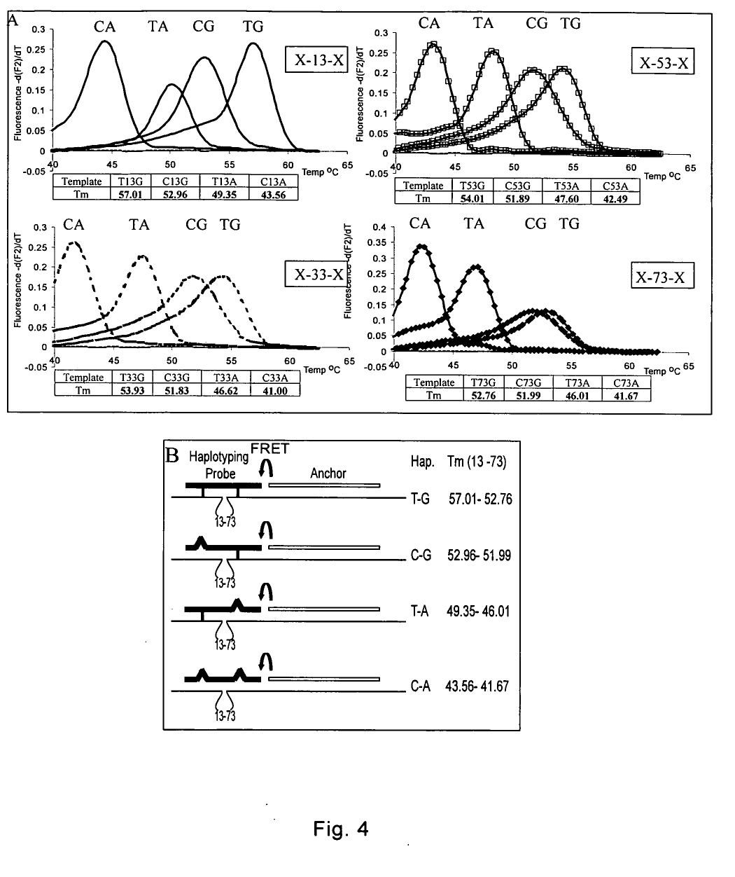

To address this, for each haplotype a series of templates with increasing length between the SNPs (FIG. 2 and FIG. 4) were hybridized with the “1537/38 hap/lpo” probe. Results of the melting analysis of the probe annealed on the 16 templates are shown in FIG. 4A. The samples are labeled as in FIG. 3 and the different lengths between the SNPs are presented on different graphs although the experiments were performed on the same run. Tms obtained for each derivative melting curve from this experience are reported for each template. The 4 haplotypes are recognizable even when the distance of untemplated nucleotide in the template is 73 nt as indicated by the example of the differences in Tms between the two most similar haplotypes (T-G and C-G) that differ only by the SNP the furthest from the point where FRET occurs (FIG. 4B).

To mimic samples heterozygous at both loci, equimolar amounts of artificial templates were mixed with the TxG and the CxA haplotypes (FIG. 5 top) or the TxA and CxG haplotypes (FIG. 5 bottom). PCR was performed and products were analyzed by recording melting of the 1537/38 hap/lpo haplotyping probe. Two derivative melting curves, corresponding to the premixed haplotypes were observed (diamond) in all cases. These are the results from the melting of the haplotyping probe hybridizing on single PCR products. Melting of a probe bridging two different PCR products would appear as derivative melting curves for the absent haplotypes. We have occasionally observed these events with the X73X. In these cases 4 curves, corresponding to the 4 haplotypes were observed (data not shown) but dilution of PCR products prior to melting should reduce these occurrences. An example of melting curves revealing intermolecular bridging reaction products is shown in FIG. 6 using the C73G and T73A artificial templates. The occurrence of these intermolecular reaction demonstrate that the probes can also be used to bridge two PCR products. We anticipate that these two products could be from different reactions that amplify different DNA sequences. Therefore the bridging would allow detection and analysis of polymorphisms found in different genes. This property could be applied to the detection of multiple polymorphisms in a panel containing at least two different PCR products.

The method described above provides a simple method to directly establish the haplotype of at least 2 polymorphisms in close proximity. The system has the advantage of being performed in a closed tube without additional manipulation of DNA after PCR and is amenable to high throughput. The system relies on the effect of mismatches on the thermodynamic stability of a nucleic acid with its template. Many applications and examples addressing single mismatches have been reported but only few data address stability of multiple mismatches. The present invention is directed to hybridization probes to establish the phases (haplotype) of two loci separated by large distances, for example, up to 80, 100, 200, 300, 400, 500 or more nucleotides.

The data specifically demonstrate that the 13 to 73 nt, present in the templates and not in the probe, bulge or loop out, during binding and that the probe dissociates as a unit. Stability of the probe/template structure might involve cooperativity along the DNA backbone, base stacking interactions and secondary structures formed inside the loop. As fluorescence due to FRET is lost, and recorded as a melting curve when the two interacting fluorophores (FITC and LCRed 640) are separated, the observation that the polymorphism located downstream of the loop (compare TG and CG or TA and CA haplotypes on FIG. 4C) affects the stability of the probe, indicates that the probe dissociates as a unit.

Comparison of the TG and T13G templates (FIG. 4C) shows that the presence of extra nucleotides in the template is sufficient to significantly reduce the stability of the probe. This data also show that the differences in size and/or sequence of the “loop out” affects the stability of the probe suggesting that relatively large insertions can be detected with hybridization probes.

### EXAMPLE 2

### Haplotyping SNPS in Close Proximity

The following example further illustrates the utility of the methods and materials of the present invention in haplotyping SNPs in close proximity. The assay described below uses properties of melting temperatures of hybridization probes covering two SNPs of interest to haplotype the beta 2 adrenergic receptor (B2AR) gene. B2AR encodes for a G protein coupled receptor that mediates the action of catecholamines and is the target for beta-agonist and beta-blockers involved in the treatment for asthma and congestive heart failure. Twelve haplotypes have been described in the human population using 13 SNPs distributed along the gene. Different drug responses have been associated with the different haplotypes (Drysdale et al., Proc Natl Acad Sci USA 97(19):10483-8 (2000)). The three most common haplotypes are distinguishable by SNPs at position −20, +46 and +79 (FIGS. 6A and 6B). Two haplotyping probe sets were designed. One overlaps the −20/+46 SNPs separated by 66 nucleotides and the other the +46/+79 SNPs separated by 33 nucleotides. Using both haplotyping probe sets we can distinguish the 3 haplotypes in a random DNA population (Pont-Kingdon and Lyon, Nucleic Acids Research, e89, 2005). This approach is useful in clinical molecular genetics diagnostics where direct haplotyping is needed rather than population statistical haplotyping approaches or complex allele specific approaches (LittleJohn et al, Human Mutation 20:479-487 (2002).

Primers and FRET Probes

The two pairs of SNPs and the primers from the B2AR gene are described by Drysdale et al., Proc Natl Acad Sci USA 97(19):10483-8 (2000). Sequences of primers were as follows:

B2AR-F1: 5′-gcagagccccgcc-3′
B2AR-R1: 5′-aaacacgatggccaggac-3′.

Sequences of probes were are given in FIG. [1C]

PCR, Melting Conditions and Analysis for Genotyping and Haplotyping

All PCR were performed in capillary tubes with 10 ul reactions on a LightCycler Instrument (Roche Applied Science) using the 5.32 run version with automated gain adjustment. PCR and haplotyping of the B2AR gene was performed in 1× LightCycler-DNA Master Hybridization Probes (Roche Applied Science), adjusted to a final MgCl₂ concentration of 3 mM with the forward and the reverse primers (B2AR-F and B2AR-R Table 2) at 0.5 μM each and the probes at 0.2 μM each. The following conditions were used for the reactions: denaturation at 95° C. for 0 seconds, annealing at 60° C. for 10 seconds and extension at 72° C. for 15 seconds for 40 cycles. Programmed transition rates were 20° C./second from denaturation to annealing and from extension to denaturation and 2° C./seconds from annealing to extension. The amplification cycles were followed by a melting cycle in which DNA was denatured by holding 30 sec at 95° C., cooling to 30° C. at the slow rate of 5° C./sec and raising the temperature to 70° C. at the rate of 0.1° C./sec. Fluorescence was continuously monitored during the melt.

Melting curves were converted into negative derivative curves of fluorescence with respect to temperature (−dF/dT) by the LightCycler Data Analysis software. All analyses were performed with background correction and color compensation. Haplotyping of B2AR-20/46 SNPs was analyzed using the F2 channel and haplotyping of B2AR 46/79 SNPs was analyzed using the F3 channel

Samples

Samples used for this study were from DNA de-identified following Institutional Review Board protocol. They were extracted from whole blood with the MagNa Pure LC DNA Isolation Kit I (Roche Applied Science)

Results

To demonstrate the ability of hybridization probes to establish haplotypes, polymorphisms were selected from the B2AR promoter (Drysdale et al., Proc Natl Acad Sci USA 97(19):10483-8 (2000); Littlejohn et al., Hum Mutat 20(6):479 (2002)). Positions −20 (T/C), +46 (A/G) and +79 (C/G) were chosen because they allow differentiation of the 3 main known haplotypes (FIG. 6A) and because the distance between the loci are in the 100 nt range (FIG. 1B). These polymorphisms are not able to distinguish the main haplotypes (#2, 4 and 6) from the less common ones (#13, 5,7, 8, 9,10, 11 &12) (FIG. 6A). The 3 polymorphisms are amplified on a 218 nt PCR fragment (FIG. 6B) and analyzed using 2 sets of hybridization probes (FIGS. 6C & 6D). These two different sets of SNPs are two additional examples showing of the ability to haplotype using hybridization probes that loop out a sequence of the template. A first set (B2AR−20/46) is composed of an anchor probe and an haplotyping probe that perfectly matches haplotype #2 except for a 55 nt internal deletion between the SNP −20 and 46. The second set analyzes the haplotypes of positions 46 and 79. The haplotyping probe is also a perfect match to haplotype #2 except the internal 22 nt not present in the probe. The 3 haplotypes were identified with both probe sets as shown in FIG. 6E (SNP −20 and 46) and 6F (SNP 46/79). In both cases haplotypes # 4 and #6 differ by the nucleotide furthest from the anchor; the difference in Tms indicate that the probe dissociated as a unit from its template.

### EXAMPLE 3

### Haplotvping of the B2AR Receptor Gene

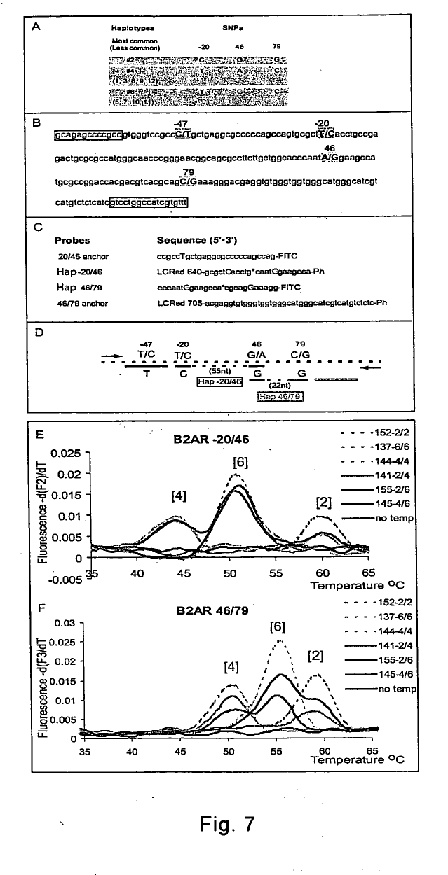

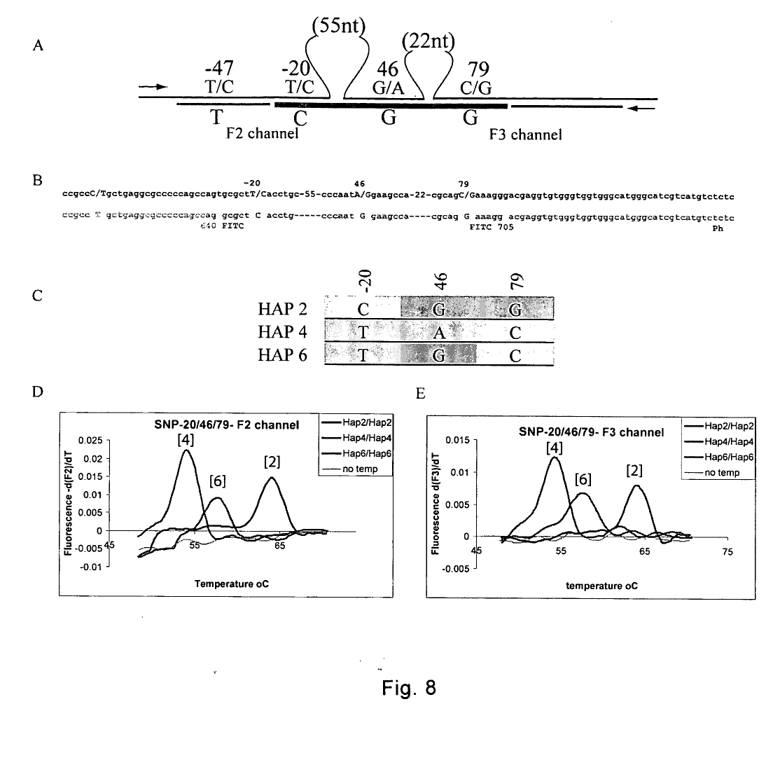

A loop out probe hybridizing with the 3 SNPs at position −20, 46 and 79 of the B2AR receptor gene was created to test the possibility of haplotyping 3 SNPs in one experiment with two sequences from the template looped out (FIG. 7A). This probe is labeled at both ends with a FITC fluorophore. It is anchored on the −20 SNP side by an oligonucleotide labeled in 3′ with LCred640 and on the 79 SNP side by an oligonucleotide labeled in 5′ with LCred705 (FIG. 7B). Melting temperature of this probe in determined both in the F2 channel (LCred640, −20 SNP side) and the F3 channel (LCred705, 79 SNP side). The probe was tested on 3 samples, each carrying 2 chromosomes with the haplotype 2 or the haplotype 4 or the haplotype 6 (FIG. 7C). Nucleotides in the probe are a perfect match with the haplotype 2, are mismatched in 2 positions with haplotype 6 and at 3 positions with haplotype 4. Data (FIGS. 7D & 7E) shows single melting curves in both channels. Melting temperature is specific for each haplotype, and identical in both channels indicating that the probe, forming 2 loops in the template, acts as a unit and is able to determine haplotypes.

### EXAMPLE 4

### Multiplex Genotyping of Beta-Globin Mutations with Ipo FRET Probe

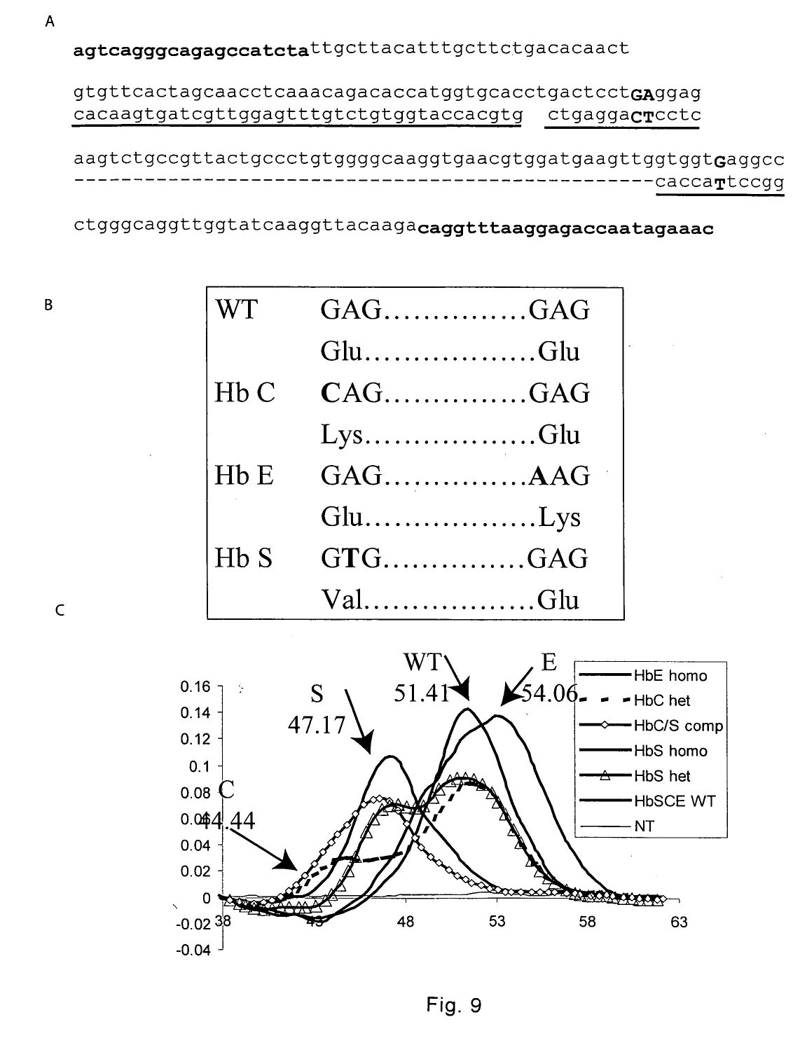

The following example illustrates the utility of the methods and materials of the present invention in simultaneously genotyping several SNPs. Mutations in the beta-globin gene are responsible for diverse malfunctions of hemoglobin. They lead to anemias with different severity. Three disease causing SNPs are found in the first exon of the beta-globin gene. Two are adjacent to each other while the third is located 58 nucleotides downstream (FIG. 9A). Mutations in these 3 SNPs are independent of each other so each genotype corresponds to a unique haplotype. Therefore, in this case, genotyping and haplotyping are equivalent. The G to C mutation at position 96 (FIG. 9B) is responsible for HbC anemia. The A to T at position 97 is responsible for the HbS anemia (sickle cell anemia) and the G to A mutation at position 156 is responsible for HbE anemia. In order to genotype all the SNPs in one step, a lpo probe that interrogate the 3 SNPs simultaneously was designed. It hybridizes perfectly with the HbE allele with the exception of 49 nucleotides omitted between positions 96/97 and 156 (FIG. 9A).

Primers and FRET Probes

Sequence of the primers are in bold in FIG. 9A. They had been described by Herrmann et al (Rapid beta-globin genotyping by multiplexing probe melting temperature and color. Clin Chem. 2000 March; 46(3):425-8. Erratum in: Clin Chem. 2004 June;50(6): 1111. Clin Chem. 2004;50(5):982.). Sequence of the probes are as follows:

lpo probe: 5′ggccttaccacctcctcaggagtc-FITC,
anchor probe: LCred 640-gtgcaccatggtgtctgtttgaggtt
gctagtgaacac-C3 blocker.

PCR and Melting Conditions

PCR was performed in 10 μl reactions in glass capillaries on a Light Cycler Instrument (Roche Applied Science). Primers concentration were 0.5 μM and probes 0.2 μM. The reaction was performed in 1× LightCycler-DNA Master Hybridization Probes (Roche Applied Science), adjusted to a final MgCl2 concentration of 3 mM. PCR was performed for 40 cycles consisting of denaturation at 95° C. for 0 sec and annealing/extension at 63° C. for 30 sec. Transition rate from denaturation to annealing/extension (and vise versa) were 20° C./sec. Melting conditions were as follow: Denaturation by holding for 5 sec at 95° C., cooling to 35° C. at a rate of 20° C./sec and holding at this temperature for 10 sec. Temperature was increased to 75° C. at a rate of 0.1° C./sec. Fluorescence was continuously monitored during the melt.

Samples.

Samples used have all been previously sequenced at the beta-globin locus. Samples were de-identified.

Results

FIG. 9C shows the distinction of the 4 different genotypes (HbE, Wild type, HbS and HbC) using melting curve analysis of the FRET lpo probe described above. Homozygous, heterozygous and compound samples can be identified by their melting profile.

### EXAMPLE 5

### Genotyping of Beta-Globin with Unlabeled lpo Probes

The following example demonstrates that unlabeled lpo probes are able to distinguish the 4 genotypes described above (Hb S, Hb C and Hb E and Wt). Unlabeled probes are therefore able to be used as FRET probe for haplotyping, multiplex genotyping and deletion detection. High resolution melting of unlabeled probes in presence double stand specific dyes have previously been described (Zhou et al., Clin Chem 50:1328-1335 (2004).

Primers and Unlabeled Probes

Sequence of the primers are as in Example 4. The sequence of the unlabeled probe is identical to the FRET lpo probe of Example 4 except that the fluorophore in 3′ is replaced with a phosphate block as follows:

Unlabeled probe: 5′ggccttaccacctcctcaggagtc-
Phosphate block.

PCR and Melting Conditions

PCR was performed in 10 μl reactions in glass capillaries on a Light Cycler Instrument (Roche Applied Science). Forward primer concentration was 5 μM, reverse primer 0.5 μM and unlabeled probe 15 μM. The reaction was performed in 1× LightCycler-DNA Master Hybridization Probes (Roche Applied Science), adjusted to a final MgCl2 concentration of 3 mM and in presence of 1× LCgreen I (Idaho Technology, Salt Lake City, Utah). PCR was performed for 60 cycles consisting of denaturation at 95° C. for 0 sec and annealing/extension at 63° C. for 30 sec. Melt was performed in a HR1 instrument (Idaho Technology, Salt Lake City, Utah) or in a Light-Scanner instrument following manufacturer recommendation. Melts were recorded from 35 to 90 Fluorescence was continuously monitored during the melt.

Samples

Homozygous samples from example 4 were used.

Results

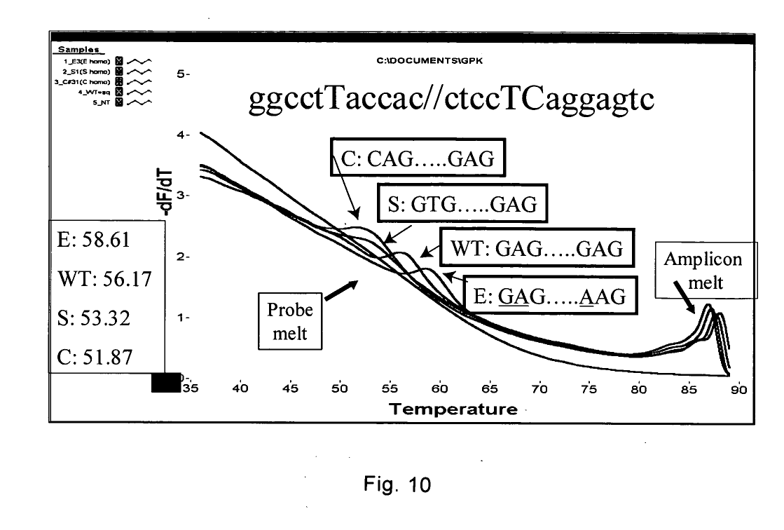

FIG. 10 shows the resolution of the 4 genotypes using an unlabeled probe and high resolution melting in a HR1 instrument. Melting derivative curves of the probe allow the identification of all 4 genotypes.

### EXAMPLE 6

### Detection of Deletion Mutations

The following examples demonstrate that lpo probes are able to detect small insertion/deletions (indels). The three biological systems presented above (WIAF 1537-1537, ADBR2 and beta-globin) are used for demonstration. In each case, melting profile of a lpo probe was compared with two complementary templates. The templates are synthesized oligonucleotides containing or not containing the sequence absent in the lpo probe. Hybridization/melting of the lpo probes from these templates mimic a situation with a lpo probe complementary to genomic sequences surrounding an insertion/deletion locus.

Templates

One set of templates is identical to the genomic sequences already presented (FIG. 1A, FIG. 7B, FIG. 9A). These sequences include the genomic sequence absent in the lpo probe and the sequence complementary to the anchor probes A loop between probe and template is expected to form. Another set of template have sequences perfectly matched to the lpo probe and its anchor. In this case no loop is formed.

Melting

Templates, lpo probes and anchor probes are mixed in equimolar ratio (0.2 μM each) in a capillary tube in 1× LightCycler-DNA Master Hybridization Probes (Roche Applied Science). No PCR is performed and the mixture is denatured (95° C. for 5 sec), annealed at a rate of 20° C./sec to 35° C. and melt to 75° C. at rates varying from 0.1 to 0.3° C./sec.

Results

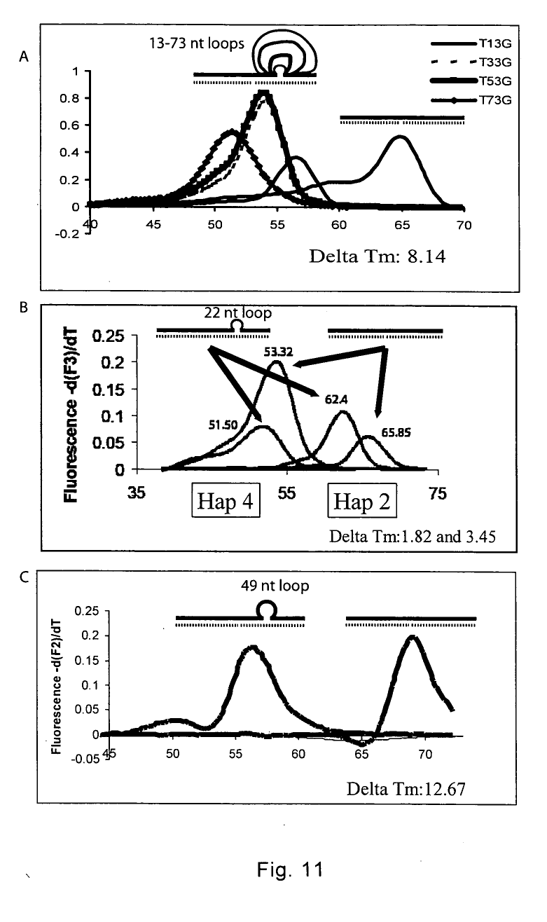

FIG. 11 presents the results obtained with WIAF 1537-1538 (FIG. 11A), ADRB2 haplotypes 2 and 4 (FIG. 11B) and beta-globin (FIG. 11C). Results show that in all cases the lpo probe is more stable when annealed on a template that does not “loop out” upon annealing. In each case the presence of additional sequences is detectable by a lower melting temperature. In the examples shown in FIGS. 10A and 10C, the destabilization of the probe due to the presence of the loop is considerable (8 to 12° C.). We concluded from these experiments that if a lpo probe is placed on a indel locus the presence of the insertion is detectable. This could for example be used for the detection of the CCR5-Δ32 allele that confers resistance to HIV.

We claim:

1. A chimeric nucleic acid probe for determining a haplotype or genotype of one or more polynucleotide templates each having at least one genetic locus characterized by multiple alleles, wherein the probe comprises two or more contiguous binding regions, each binding region encompassing a genetic locus and being capable of hybridizing to corresponding non-contiguous binding regions of the polynucleotide templates.

2. The nucleic acid probe according to claim 1, wherein at least two binding regions of the probe are capable of hybridizing to corresponding non-contiguous binding regions located on separate nucleic acid templates.

3. The nucleic acid probe according to claim 1, wherein the binding regions of the probe are capable of hybridizing to corresponding non-contiguous binding regions located on a single nucleic acid template.

4. The nucleic acid probe according to claim 3, wherein the binding regions of the probe correspond to binding regions on the nucleic acid template that include genetic loci characterized by multiple alleles that define a haplotype.

5. The nucleic acid probe according to claim 3, wherein the binding regions of the probe correspond to binding regions on the nucleic acid template that encompass genetic loci characterized by multiple alleles that define a haplotype, wherein one of the genetic loci is located between the template binding regions and is characterized by an allele consisting of an insertion or deletion mutation.

6. The nucleic acid probe according to claim 1, wherein at least one binding region of a polynucleotide template comprises a genetic locus characterized by multiple alleles.

7. The nucleic acid probe according to claim 1, wherein at least two binding regions of the polynucleotide templates comprise a genetic locus characterized by multiple alleles.

8. The nucleic acid complex according to claim 12, wherein at least a portion of the binding region of the probes is complementary to one allele of the genetic locus within the binding region of the polynucleotide templates.

9. A nucleic acid complex for determining a haplotype or genotype of one or more polynucleotide templates each having at least one genetic locus characterized by multiple alleles, wherein the complex comprises a nucleic acid probe hybridized to a polynucleotide template, wherein the probe comprises two or more contiguous binding regions, each binding region encompassing a genetic locus and being capable of hybridizing to corresponding non-contiguous binding regions of the polynucleotide templates.

10. The nucleic acid complex according to claim 9, wherein at least two binding regions of the probe are capable of hybridizing to corresponding non-contiguous binding regions located on separate nucleic acid templates.

11. The nucleic acid complex according to claim 9, wherein the binding regions of the probe are capable of hybridizing to corresponding non-contiguous binding regions located on a single nucleic acid template.

12. The nucleic acid complex according to claim 11, wherein the binding regions of the probe correspond to binding regions on the nucleic acid template that include genetic loci characterized by multiple alleles that define a haplotype.

13. The nucleic acid complex according to claim 11, wherein the binding regions of the probe correspond to binding regions on the nucleic acid template that encompass genetic loci characterized by multiple alleles that define a haplotype, wherein one of the genetic loci is located between the template binding regions and is characterized by an allele consisting of an insertion or deletion mutation.

14. A nucleic acid complex according to claim 9, wherein at least one binding region of a polynucleotide template comprises a genetic locus characterized by multiple alleles.

15. The nucleic acid complex according to claim 9, wherein at least two binding regions of the polynucleotide templates comprise a genetic locus characterized by multiple alleles.

16. The nucleic acid complex according to claim 9, wherein at least a portion of the binding region of the probes is complementary to one allele of the genetic locus within the binding region of the polynucleotide templates.

17. A method for determining a haplotype or genotype of one or more polynucleotide templates each having at least one genetic locus characterized by multiple alleles, comprising:
(a) providing a nucleic acid probe comprising two or more contiguous binding regions, each binding region encompassing a genetic locus and being capable of hybridizing to corresponding non-contiguous binding regions of the polynucleotide templates;
(b) hybridizing the probe to the alleles to form a complex of the probe and the alleles;
(c) dissociating the complex and determining the melting curve profile of the complex; and
(d) correlating the melting curve profile of the complex with a melting curve profile characteristic of the alleles at each genetic loci, thereby determining the haplotype or genotype of the allele at each genetic loci.

18. The method according to claim 17, wherein at least two binding regions of the probe are capable of hybridizing to corresponding non-contiguous binding regions located on separate nucleic acid templates.

19. The method according to claim 17, wherein the binding regions of the probe are capable of hybridizing to corresponding non-contiguous binding regions located on a single nucleic acid template.

20. The method according to claim 19, wherein the binding regions of the probe correspond to binding regions on the nucleic acid template that include genetic loci characterized by multiple alleles that define a haplotype.

21. The method according to claim 19, wherein the binding regions of the probe correspond to binding regions on the nucleic acid template that encompass genetic loci characterized by multiple alleles that define a haplotype, wherein one of the genetic loci is located between the template binding regions and is characterized by an allele consisting of an insertion or deletion mutation.

22. A method according to claim 17, wherein at least one binding region of a polynucleotide template comprises a genetic locus characterized by multiple alleles.

23. The nucleic acid complex according to claim 17, wherein at least two binding regions of the polynucleotide templates comprise a genetic locus characterized by multiple alleles.

24. The nucleic acid complex according to claim 17, wherein at least a portion of the binding region of the probes is complementary to one allele of the genetic locus within the binding region of the polynucleotide templates.

25. A method according to claim 17, wherein at least one genetic locus is characterized by an allele consisting of an insertion or deletion mutation flanked by the binding regions of the template.

26. The method according to claim 25, wherein at least one of the binding regions of the probe corresponds to a binding regions on the nucleic acid template that includes a genetic locus characterized by multiple alleles that define a haplotype with the insertion or deletion.

27. The method according to claim 25, wherein the binding regions of the probe correspond to binding regions on the nucleic acid template that include genetic loci characterized by multiple alleles that define a haplotype with the insertion or deletion.
